(19) United States
(12) Patent Application Publication
(10) Pub. No.: US 2026/0295265 A1
(43) Pub. Date: Oct. 1, 2026

(54) CONTROLLING ELECTRICAL STIMULATION BY MULTIPLE MEDICAL DEVICES

(71) Applicant: Medtronic, Inc., Minneapolis, MN (US)

(72) Inventors: Todd D. Zenisek, Georgetown, TX (US); Joshua J. Nedrud, Roseville, MN (US); Andrew J. Cleland, Macquarie Park, New South Wales (AU); Nathan A. Torgerson, Andover, MN (US); Scott R. Stanslaski, Shoreview, MN (US)

(21) Appl. No.: 19/479,367

(22) PCT Filed: Apr. 11, 2024
(86) PCT No.: PCT/IB2024/053542

§ 371 Date: Oct. 28, 2025

Related U.S. Application Data
(60) Provisional application No. 63/498,988, filed on Apr. 28, 2023.

(51) Int. Cl. A61N 1/36; A61N 1/372

(52) U.S. Cl. CPC A61N 1/36139; A61N 1/36021; A61N 1/36062; A61N 1/36132; A61N 1/3615; A61N 1/36171; A61N 1/36175; A61N 1/37288

(57) ABSTRACT

This disclosure is directed to devices, systems, and techniques for controlling electrical stimulation therapy. In some examples, a system includes: a first implantable medical device (IMD) implanted within a first tissue of a patient. The first IMD may include: sensing circuitry configured to generate sensed information representative of an evoked electrical signal elicited by a first stimulation signal delivered from a second IMD different from the first IMD to a second tissue of the patient, the second tissue being at a different location than the first tissue; and processing circuitry configured to: determine, based on the sensed information representative of the evoked electrical signal, one or more stimulation parameters at least partially defining a second stimulation signal; and control stimulation generation circuitry of the first IMD to deliver the second stimulation signal to the first tissue of the patient according to the one or more stimulation parameters.

This Application claims priority from U.S. Provisional Patent Application 63/498,988, filed 28 Apr. 2023, the entire content of which is incorporated herein by reference.

### TECHNICAL FIELD

This disclosure generally relates to electrical stimulation therapy, and more specifically, control of electrical stimulation therapy delivered via multiple devices.

### BACKGROUND

Medical devices may be external or implanted and may be used to deliver electrical stimulation therapy to patients via various tissue sites to treat a variety of symptoms or conditions such as chronic pain, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Stimulation proximate the spinal cord, proximate the sacral nerve, within the brain, and proximate peripheral nerves are often referred to as spinal cord stimulation (SCS), sacral neuromodulation (SNM), deep brain stimulation (DBS), and peripheral nerve stimulation (PNS), respectively.

### SUMMARY

In general, the disclosure is directed to devices, systems, and techniques for controlling electrical stimulation therapy by sensing at least one of stimulation signals or evoked electrical signals, such as evoked compound action potentials (ECAPs). A medical device (e.g., an implantable medical device) can deliver one or more stimulation signals (e.g., one or more pulses) to the patient via one or more leads, and the medical device may sense the respective delivered stimulation signals and/or ECAPs elicited by the stimulation signals. For example, the medical device may be configured to sense electrical signals via one or more electrodes.

In some examples, multiple medical devices (e.g., a first medical device and a second medical device) can deliver different stimulation signals, e.g., to different parts of the body of the patient. The effects of the different stimulation signals may lead to unintended interference or other effects to at least one of the stimulation signals by another of the stimulation signals. This disclosure describes example systems, devices, and methods for coordinating the timing, amplitude, frequency, or other parameters that define the delivery of stimulation signals by a plurality of medical devices to the patient. coordination of the timing of the stimulation signals may prevent unintended interference or other effects between the stimulation signals, thereby increasing the efficacy of each stimulation signal (e.g., reduction in symptoms and/or reduction in side effects). In some examples, coordination of the timing of the stimulation signals may propagate, amplify, or diminish one or more of the stimulation signals traveling through the body of the patient. The propagation, amplification, or diminishment of a stimulation signal may increase the efficacy, and/or other medical effects of the stimulation signal.

In some examples, the disclosure describes a system comprising: a first implantable medical device (IMD) implanted within a first tissue of a patient, the first IMD comprising; sensing circuitry configured to generate sensed information representative of an evoked electrical signal elicited by a first stimulation signal delivered from a second IMD different from the first IMD to a second tissue of the patient, the second tissue being at a different location than the first tissue; and processing circuitry configured to: determine, based on the sensed information representative of the evoked electrical signal, one or more stimulation parameters at least partially defining a second stimulation signal; and control stimulation generation circuitry of the first IMD to deliver the second stimulation signal to the first tissue of the patient according to the one or more stimulation parameters.

In some examples, the disclosure describes a method comprising: sensing, via sensing circuitry of a first implantable medical device (IMD) implanted within a first tissue of a first region of a patient, information representative of an evoked electrical signal elicited by a first stimulation signal delivered from a second IMD different from the first IMD to a second tissue of a second region of the patient; determining, by processing circuitry of the first IMD and based on the sensed information representative of the evoked electrical signal, one or more stimulation parameters at least partially defining a second stimulation signal; and controlling, by the processing circuitry, stimulation generation circuitry of the first IMD to deliver the second stimulation signal to the first tissue according to the one or more stimulation parameters.

In some examples, the disclosure describes a computer-readable medium comprising instructions that, when executed, cause processing circuitry of an implantable medical device (IMD) to sensing, via sensing circuitry of a first implantable medical device (IMD) implanted within a first tissue of a first region of a patient, information representative of an evoked electrical signal elicited by a first stimulation signal delivered from a second IMD different from the first IMD to a second tissue of a second region of the patient; determining, based on the sensed information representative of the evoked electrical signal, one or more stimulation parameters at least partially defining a second stimulation signal; and controlling stimulation generation circuitry of the first IMD to deliver the second stimulation signal to the first tissue according to the one or more stimulation parameters.

In some examples, the disclosure describes a system comprising: an external device comprising: communications circuitry configured to be in communications with a first implanted medical device (IMD) implanted within a first tissue of a patient and a second IMD implanted within a second tissue of the patient, the second tissue being at a different location than the first tissue; and processing circuitry configured to: receive, via the communications circuitry and from the second IMD, stimulation information representative of a first stimulation signal delivered from the second IMD to the second tissue; receive, via the communications circuitry and from the first IMD, sensed information representative of an evoked electrical signal elicited by the first stimulation signal; determine, based on the received stimulation information and the received sensed information, one or more stimulation parameters at least partially defining a second stimulation signal; and transmit, via the communications circuitry, instructions to the first IMD to deliver the second stimulation signal to the first tissue according to the one or more stimulation parameters.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example system that includes a plurality of implantable medical devices (IMDs) configured to deliver stimulation therapy to a patient and an external programmer, in accordance with one or more techniques of this disclosure.

FIG. 2 is a block diagram illustrating an example configuration of components of an IMD of FIG. 1, in accordance with one or more techniques of this disclosure.

FIG. 3 is a block diagram illustrating an example configuration of components of the external programmer of FIG. 1, in accordance with one or more techniques of this disclosure.

FIG. 4 is a graph illustrating an example timing diagram for the delivery of stimulation pulses by the plurality of IMDs of FIG. 1, in accordance with one or more techniques of this disclosure.

FIG. 5A is a graph illustrating example evoked compound action potentials (ECAPs) sensed for respective stimulation pulses, in accordance with one or more techniques of this disclosure.

FIG. 5B is an example timing diagram illustrating an example of electrical stimulation pulses, respective stimulation pulses, and respective sensed ECAPs, in accordance with one or more techniques of this disclosure.

FIG. 6 is a flow diagram illustrating an example process for controlling simulation based on a plurality of stimulation pulses by system including a plurality of IMDs, in accordance with one or more techniques of this disclosure.

FIG. 7 is a flow diagram illustrating an example process for adjusting a simulation pulse based on changes in a sensed ECAP signal, in accordance with one or more techniques of this disclosure.

FIG. 8 is a flow diagram illustrating another example process for controlling stimulation based on a plurality of stimulation signals by system including a plurality of IMDs, in accordance with one or more techniques of this disclosure.

Like reference characters denote like elements throughout the description and figures.

### DETAILED DESCRIPTION

The disclosure describes examples of medical devices, systems, and techniques for adjusting electrical stimulation therapy delivered to a patient by adjusting timing of one or more of a plurality of stimulation signals generated by a plurality of medical devices, e.g., based on stimulation signals and/or evoked electrical signals (e.g., evoked compound action potentials (ECAPs)) sensed or detected by a medical device. Electrical stimulation therapy is typically delivered by a medical device to a target tissue (e.g., one or more nerves or muscle) of a patient via two or more electrodes. Parameters of the electrical stimulation therapy (e.g., electrode combination, voltage or current amplitude, pulse width, pulse frequency, etc.) are selected by a clinician and/or the patient to provide relief from various symptoms, such as pain, muscle disorders, etc. In some examples, the patient may receive electrical stimulation therapies, each therapy being delivered by a different medical device of a plurality of medical devices. The plurality of medical devices may be of the same type (e.g., spinal cord stimulators (SCS), peripheral nerve stimulators (PNS), or of different types.

ECAPs are a measure of neural recruitment because each ECAP signal represents the superposition of electrical potentials generated from axons firing in response to an electrical stimulus (e.g., a stimulation pulse). Changes in a characteristic (e.g., an amplitude of a portion of the signal, an area under one or more peaks, frequency content, and/or maximum and/or minimum peak timing) of an ECAP signal occur as a function of how many axons have been activated by the delivered stimulation pulse. A system can monitor changes in the characteristic of the ECAP signal and use that change in the characteristic to adjust one or more stimulation parameters that at least partially defines the stimulation pulses delivered to the patient. For example, the system can reduce the intensity of stimulation pulses (e.g., reduce a current amplitude and/or pulse width) in response to detecting an increase in an amplitude of an ECAP signal.

A medical device may be configured to sense stimulation signals and adjust stimulation parameters based on a characteristic of one or more stimulation signals. Even if an ECAP signal is not detectable, the medical device may detect one or more stimulation signals caused by the stimulation pulse. The medical device may determine or adjust values of one or more parameters that at least partially define stimulation pulses based on characteristics of the detected stimulation signals. For example, the medical device may adjust timing parameters of stimulation pulses based at least in part on the detected stimulation signals. In some examples, the stimulation signals may include information indicative of a distance between multiple medical devices along the nerves of the patient. In this way, one medical device may determine a distance to another medical device and adjust the timing parameters of a corresponding stimulation pulse to be delivered by the one medical device, e.g., to synchronize the stimulation pulses generated by both medical devices or to propagate, amplify, diminish, or cancel out one or more of the stimulation pulses and/or one or more action potential pulses, e.g., to improve efficacy of the stimulation therapy and/or deliver a desired stimulation therapy to the patient.

The term “stimulation signal” may be used herein to describe a signal that a second medical device senses in response to a stimulation pulse delivered by a first medical device. An electrical potential of stimulation electrodes of the first medical device during a window of time in which the first medical device delivers the stimulation pulse may cause the sensing circuitry of the second medical device to generate a sense signal which is representative of the stimulation pulse delivered during the window of time. The stimulation signal is thus representative of electrical potential changes in tissue directly caused by the delivered stimulation pulse. Conversely, an ECAP is a signal representative of physiological action (e.g., depolarizing nerve fibers) caused by the stimulation pulse. An ECAP represents a detected physiological response to a stimulation pulse, and a stimulation signal represents the direct detection of the stimulation pulse itself and associated changes in the charge in tissue.

In some examples, the medical device may deliver stimulation pulses in the form of control pulses and informed pulses. More specifically, electrical stimulation pulses are delivered in the form of informed pulses and control pulses that are at least partially interleaved with each other. Control pulses (e.g., stimulation signal test pulses) are those stimulation pulses that are configured to elicit one or both of a stimulation signal and a detectable ECAP signal. In some examples, control pulses may contribute to the therapy for a patient. In other examples, control pulses do not contribute to the therapy for the patient, e.g., non-therapeutic pulses. In this manner, control pulses may or may not be configured to elicit a therapeutic effect for the patient. Informed pulses are those stimulation pulses that are at least partially defined by one or more parameters based on the detectable stimulation signal elicited from one or more control pulses. In some examples, one or more informed pulses are at least partially defined by one or more parameters based on a respective ECAP elicited from one or more control pulses. In this manner, the informed pulses are “informed” by the ECAP signal detected from a control pulse. Informed pulses are also configured to provide a therapy to a patient, such as paresthesia that relieves pain symptoms.

As described herein, a second medical device may detect ECAP signals and/or stimulation signals from a first stimulation pulse delivered by the first medical device and adjust parameters of a second stimulation pulse configured to be delivered by the second medical device accordingly. In some examples, the second medical device may adjust the parameters of the second stimulation pulse to synchronize delivery of the first stimulation pulse and the second stimulation pulse by the first medical device and the second medical device, respectively. In some examples, the second medical device may adjust the timing parameters (e.g., start of delivery, duty cycle, frequency) of the second stimulation pulse to propagate, amplify, diminish, or cancel out the first stimulation pulse. The first medical device and the second medical device be connected to different portions of the body of the patient and may communicate with each other and/or a third device (e.g., a programmer, an external device, another medical device).

The techniques of this disclosure may provide one or more advantages. For example, synchronizing multiple medical devices to deliver stimulation pulses simultaneously prevent adverse interference (e.g., destructive interference) caused by the interactions of the multiple stimulation pulses, thereby improving the efficacy of the stimulation therapy. In some examples, coordinating the timing of stimulation pulses, e.g., to alternate the delivery of stimulation pulses by multiple medical devices, may reduce power consumption by each of the medical devices and/or increase the operational lifespan of each of the medical devices. In some examples, coordinating the timing of stimulation pulses to amplify, propagate, diminish, or cancel out particular stimulation pulses may increase therapy efficacy and patient comfort. The coordination of the timing of stimulation signals from different devices may be intended to improve the efficacy of treatment targeting a single condition treated by the multiple devices or maintain efficacy the individual treatments provided by each medical device to different parts of the body when the stimulation from at least one device affects the stimulation delivered by another device.

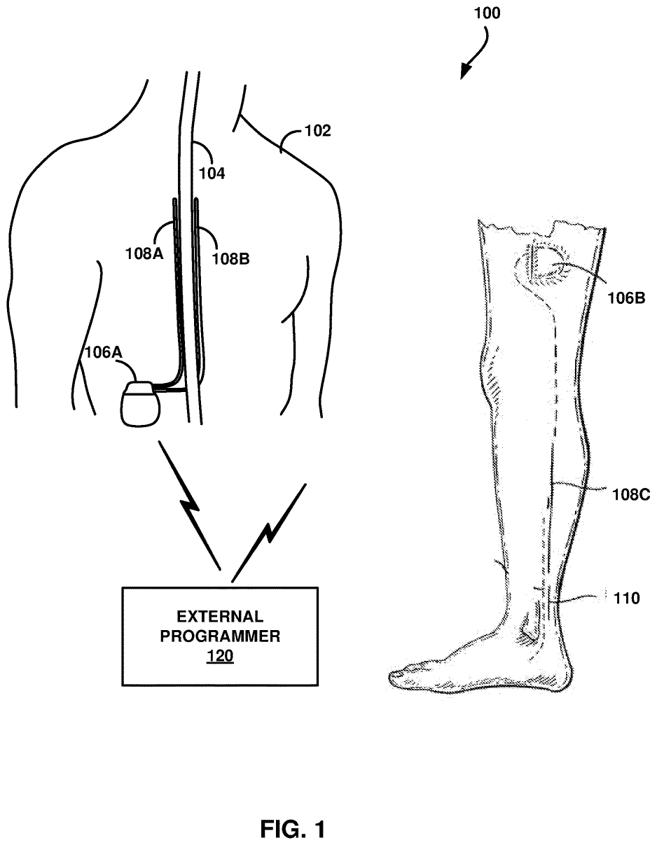

FIG. 1 is a conceptual diagram illustrating an example system 100 that includes a first implantable medical device (IMD) 106A and a second IMD 106B. While this disclosure is described primarily with reference to two IMDs 106A, 106B (collectively referred to as “IMDs 106”), other examples may include three or more IMDs 106. Each of IMDs 106 may deliver electrical stimulation pulses to tissue of the same patient 102. In some examples, at least one or more of IMDs 106 may be configured to sense stimulation signals and/or ECAPs, deliver other medical therapy to patient 102 (e.g., a therapeutic substance via a pump), and/or perform any other medical functions.

As illustrated in FIG. 1, first IMD 106A is configured to deliver spinal cord stimulation (SCS) therapy and second IMD 106B is configured to deliver peripheral nerve stimulation (PNS) therapy. In some examples, each of IMDs 106 may be configured to deliver SCS therapy and/or PNS therapy. For example, first IMD 106A and second IMD 106B may deliver separate stimulation pulses to different target tissue of patient 102 as a part of one SCS therapy. In some examples, one of IMDs 106 (e.g., first IMD 106A) may deliver “priming” stimulation pulses to glial cells of spinal cord 104 and another of IMDs 106 (e.g., second IMD 106B) may deliver “base” stimulation pulses to a peripheral nerve as a part of a SCS therapy to reduce pain experienced by patient 102. In such examples, the base stimulation pulses may generate action potentials (e.g., ECAPs) in the peripheral nerves which may be detected by one or more of IMDs 106, e.g., to adjust the stimulation parameters of stimulation pulses configured to be delivered by one or more of IMDs 106.

Each of IMDs 106 may be configured to communicate with an external programmer 120 (e.g., the same programmer or a different programmer), in accordance with one or more techniques of this disclosure. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of medical devices.

As shown in FIG. 1, system 100 includes first IMD 106A, second IMD 106B, leads 108A, 108B, and 108C (collectively referred to as “leads 108”), and external programmer 120 shown in conjunction with a patient 102, who is ordinarily a human patient. In the example of FIG. 1, first IMD 106A is an implantable electrical stimulator that is configured to generate and deliver electrical stimulation therapy to patient 102 via one or more electrodes of electrodes of leads 108A and/or 108B, e.g., for relief of chronic pain or other symptoms. Similarly, second IMD 106B is an implantable electrical stimulator that is configured to generate and deliver electrical stimulation therapy to patient 102 via one or more electrode of electrodes of lead 108C. In some examples, each of IMDs 106 may be coupled to a single lead carrying multiple electrodes or two or more leads each carrying multiple electrodes. This electrical stimulation may be delivered in the form of stimulation pulses. In some examples, IMDs 106 may be configured to generate and deliver stimulation pulses to include control pulses configured to elicit ECAP signals and/or cause IMDs 106 to sense stimulation signals. The control pulses may or may not contribute to therapy in some examples. In some examples, IMDs 106 may, in addition to control pulses, deliver informed pulses that contribute to the therapy for the patient, but which do not elicit detectable ECAPs or cause IMDs 106 to detect every phase of responsive stimulation signals. One or more of IMDs 106 may be a chronic electrical stimulator that remains implanted within patient 102 for weeks, months, or even years. In other examples, one or more of IMDs 106 may be a temporary, or trial, stimulator used to screen or evaluate the efficacy of electrical stimulation for chronic therapy. Each of IMDs 106 may be implanted within patient 102 or coupled to percutaneously implanted leads. In some examples, one or more of IMDs 106 is leadless.

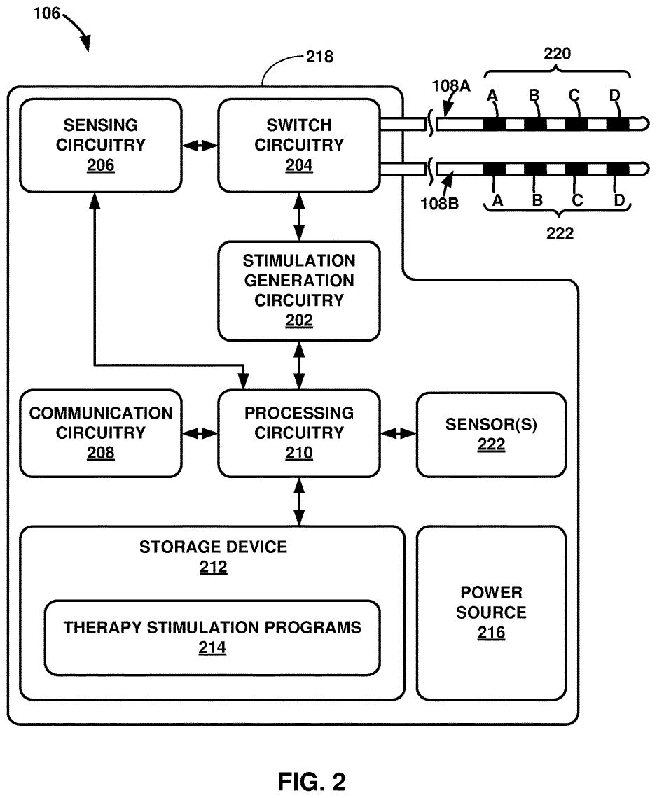

Each of IMDs 106 may be constructed of any polymer, metal, or composite material sufficient to house the components of IMD 106 (e.g., components illustrated in FIG. 2) within patient 102. In this example, Each of IMDs 106 may be constructed with a biocompatible housing, such as titanium or stainless steel, or a polymeric material such as silicone, polyurethane, or a liquid crystal polymer. One or more of IMDs 106 may be surgically implanted at a site in patient 102 near the pelvis, abdomen, thigh, or buttocks. In other examples, One or more of IMDs 106 may be implanted within other suitable sites within patient 102, which may depend, for example, on the target site within patient 102 for the delivery of electrical stimulation therapy (e.g., alone spinal cord 104 of patient 102, in leg 110 of patient 102). The outer housing of each of IMDs 106 may be configured to provide a hermetic seal for components, such as a rechargeable or non-rechargeable power source. In addition, in some examples, the outer housing of each of IMDs 106 is selected from a material that facilitates receiving energy to charge the rechargeable power source.

Electrical stimulation energy, which may be constant current or constant voltage-based pulses, for example, is delivered from each of IMDs 106 to one or more corresponding target tissue sites of patient 102 via one or more electrodes (not shown) of corresponding implantable leads 108. In the example of FIG. 1, each of leads 108 carry electrodes that are placed adjacent to the target tissue (e.g., near spinal cord 104, in leg 110). Other tissue locations may be used in other examples. One or more of the electrodes may be disposed at a distal tip of each of leads 108 and/or at other positions at intermediate points along the lead. Leads 108 may be implanted and coupled to IMD 106. The electrodes may transfer electrical stimulation generated by an electrical stimulation generator in IMD 106 to tissue of patient 102. Although leads 108 may each be a single lead, leads 108 may include a lead extension or other segments that may aid in implantation or positioning of leads 108. In some other examples, one or more of IMDs 106 may be a leadless stimulator with one or more arrays of electrodes arranged on a housing of the stimulator rather than leads that extend from the housing. In addition, in some other examples, system 100 may include one lead or more than two leads, each coupled to one of IMDs 106 and directed to similar or different target tissue sites.

The electrodes of leads 108 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 108 will be described for purposes of illustration.

The deployment of electrodes via leads 108 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 108 are linear leads having 8 ring electrodes along the axial length of the lead. In another example, the electrodes are segmented rings arranged in a linear fashion along the axial length of the lead and at the periphery of the lead.

The stimulation parameter of a therapy stimulation program that defines the stimulation pulses of electrical stimulation therapy by each of IMDs 106 through the electrodes of leads 108 may include information identifying which electrodes have been selected for delivery of stimulation according to a stimulation program, the polarities of the selected electrodes, i.e., the electrode combination for the program, and voltage or current amplitude, pulse frequency, pulse width, pulse shape of stimulation delivered by the electrodes. These stimulation parameters of stimulation pulses (e.g., control pulses and/or informed pulses) are typically predetermined parameter values determined prior to delivery of the stimulation pulses (e.g., set according to a stimulation program). However, in some examples, system 100 changes one or more parameter values automatically based on one or more factors or based on user input.

In some examples, the stimulation parameters of each therapy stimulation program may define timing parameters for the delivery of the stimulation pulses of the therapy stimulation program. The timing parameters may include, but are not limited to, a duty cycle of the stimulation pulses, a frequency of the stimulation pulses, a start time of each stimulation pulse, an end time of each stimulation pulse, or the like. The timing parameters of the different stimulation pulses of the therapy stimulation program executed by IMDs 106 may be synchronized, or may be offset by predetermined intervals.

Although FIG. 1 is directed to SCS therapy and/or PNS therapy, e.g., used to treat pain, in other examples system 100 may be configured to treat any other condition that may benefit from electrical stimulation therapy. For example, system 100 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 100 may be configured to provide therapy taking the form of deep brain stimulation (DBS), peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 102.

In some examples, leads 108 includes one or more sensors configured to allow IMD 106 to monitor one or more parameters of patient 102, such as patient activity, pressure, temperature, or other characteristics. The one or more sensors may be provided in addition to, or in place of, therapy delivery by leads 108. The one or more sensors of each of IMDs 106 may sense, e.g., from nerves of patient 102, stimulation signals and/or ECAP signals from the other of IMDs 106. For example, first IMD 106A may sense, via leads 108A and 108B and from nerves of patient 102, stimulation signals and/or ECAP signals corresponding to stimulation pulses from second IMD 106B. Similarly, second IMD 106B may sense, via lead 108C, stimulation signals and/or ECAP signals corresponding to stimulation pulses from first IMD 106A. The sensors configured to sense electrical signals may be one or more electrodes located on a lead 18 and/or sensing circuitry within IMD 106A. The sensing electrodes may be the same or different than electrodes used to deliver stimulation.

Each of IMDs 106 may be configured to deliver electrical stimulation therapy to patient 102 via selected combinations of electrodes carried by one or both of leads 108, alone or in combination with an electrode carried by or defined by an outer housing of IMD 106. The target tissue for the electrical stimulation therapy may be any tissue affected by electrical stimulation, which may be in the form of electrical stimulation pulses or continuous waveforms. In some examples, the target tissue includes nerves, smooth muscle or skeletal muscle. In the example illustrated by FIG. 1, e.g., with respect to first IMD 106A, the target tissue is tissue proximate spinal cord 104, such as within an intrathecal space or epidural space of spinal cord 104, or, in some examples, adjacent nerves that branch off spinal cord 104. Leads 108 (e.g., leads 108A, 108B) may be introduced into spinal cord 104 in via any suitable region, such as the thoracic, cervical or lumbar regions. Stimulation of spinal cord 104 may, for example, prevent pain signals from traveling through spinal cord 104 and to the brain of patient 102. Patient 102 may perceive the interruption of pain signals as a reduction in pain and, therefore, efficacious therapy results. In other examples, stimulation of spinal cord 104 may produce paresthesia which may be reduce the perception of pain by patient 102, and thus, provide efficacious therapy results.

A user, such as a clinician or patient 102, may interact with a user interface of an external programmer 120 to program and/or to control one or more of IMDs 106. Programming of one or more of IMDs 106 may refer generally to the generation and transfer of commands, programs, or other information to control the operation of one or more of IMDs 106. In this manner, Each of IMDs 106 may receive the transferred commands and programs from external programmer 120 to control electrical stimulation therapy (e.g., informed pulses) and control stimulation (e.g., control pulses). For example, external programmer 120 may transmit therapy stimulation programs, stimulation parameter adjustments, therapy stimulation program selections, user input, or other information to control the operation of each of IMDs 106, e.g., by wireless telemetry or wired connection. As described herein, stimulation delivered to the patient may include control pulses, and, in some examples, stimulation may include control pulses and informed pulses.

In some cases, external programmer 120 may be characterized as a physician or clinician programmer if it is primarily intended for use by a physician or clinician. In other cases, external programmer 120 may be characterized as a patient programmer if it is primarily intended for use by a patient. A patient programmer may be generally accessible to patient 102 and, in many cases, may be a portable device that may accompany patient 102 throughout the patient's daily routine. For example, a patient programmer may receive input from patient 102 when the patient wishes to terminate or change electrical stimulation therapy. In general, a physician or clinician programmer may support selection and generation of programs by a clinician for use by one or more of IMDs 106, whereas a patient programmer may support adjustment and selection of such programs by a patient during ordinary use. In other examples, external programmer 120 may include, or be part of, an external charging device that recharges a power source of one or more of IMDs 106. In this manner, a user may program and charge one or more of IMDs 106 using one device, or multiple devices.

As described herein, information may be transmitted between external programmer 120 and one or more of IMDs 106. Therefore, one or more of IMDs 106 and external programmer 120 may communicate via wireless communication using any techniques known in the art. Examples of communication techniques may include, for example, radiofrequency (RF) telemetry and inductive coupling, but other techniques are also contemplated. In some examples, external programmer 120 includes a communication head that may be placed proximate to the patient's body near the one or more of IMDs 106 implant sites to improve the quality or security of communication between the one or more of IMDs 106 and external programmer 120. Communication between external programmer 120 and one or more of IMDs 106 may occur during power transmission or separate from power transmission. In some examples, one or more IMDs 106 and external programmer 120 may communicate via a intermediate device (e.g., a third IMD, a body band, a wearable intermediary device such as a smartwatch or a smartphone). In some examples, IMDs 106 may be coupled in a controller/responder configuration, where first IMD 106A is configured to communicate with external programmer 120 and controls second IMD 106B.

In some examples, the intermediate device may receive input from IMDs 106 and control the delivery of electrical stimulation therapy by one or more of IMDs 106 based on the receive input signals. The intermediate device may control the delivery of electrical stimulation therapy to synchronize or desynchronize different stimulation signals delivered by IMDs 106, e.g., to amplify, propagate, diminish, or cancel out one or more of the delivered stimulation signals. The intermediate device may control IMDs 106 based on received instructions from external programmer 120 or based on instructions stored in the memory of the intermediate device. The received input signals may include, but are not limited to, evoked signals such as compound action potential (ECAP) signals or evoked resonant neural activity (ERNA) signals, local field potential (LFP) signals, electrocardiography (ECG) signals, electromyography (EMG) signals, or other signals indicative of nerve activity of patient 102. The intermediate device may cause one or more of IMDs 106 to cycle between stimulation signals, temporarily pausing the delivery of stimulation signals, adjusting the frequency of stimulation signals, adjusting the pulse widths of stimulation signals, and/or adjusting the amplitude of stimulation signals to amplify, propagate, diminish, or cancel out another stimulation signal delivered to patient 102 by another of IMDs 106.

In some examples, the intermediate device determines that one or more of IMDs 106 is detecting input signals with a higher amplitude than a baseline amplitude and causes the one or more IMDs 106 to deliver stimulation signals to patient 102 at target location(s) corresponding to the one or more IMDs 106. In some examples, the intermediate device determines, based on the input signals or feedback from patient 102, that two or more previously synchronized IMDs 106 have become desynchronized. The intermediate device may adjust the electrical stimulation signals delivered by at least one of the two or more IMDs 106 to re-synchronize electrical stimulation signals delivered by the two or more IMDs 106. The intermediate device may re-synchronize the electrical stimulation signals may adjusting the pulse width of one of the stimulation signals and/or temporarily pausing the delivery of the stimulation signal.

In some examples, one or more of IMDs 106, in response to commands from external programmer 120, delivers electrical stimulation therapy according to a plurality of therapy stimulation programs to a target tissue site in patient 102 via electrodes (not depicted) on leads 108. In some examples, one or more of IMDs 106 modifies therapy stimulation programs as therapy needs of patient 102 evolve over time and/or based on the sensed stimulation signals and/or ECAP signals from another of IMDs 106. For example, the modification of the therapy stimulation programs may cause the adjustment of at least one parameter of the plurality of informed pulses. When patient 102 receives the same therapy for an extended period, the efficacy of the therapy may be reduced. In some cases, parameters of the plurality of informed pulses may be automatically updated. In some examples, one or more IMDs 106 may become desynchronized from another of IMDs 106. In such examples, the one or more IMDs 106 may adjust stimulation parameters based on the sensed stimulation signals and/or ECAP signals, e.g., to resynchronize IMDs 106.

In the example of FIG. 1, one or more of IMDs 106 is described as performing a plurality of processing and computing functions. For example, first IMD 106A may receive the sensed stimulation signals and/or ECAP signals corresponding to a first stimulation pulse from second IMD 106B, adjust stimulation parameters (e.g., timing parameters) of a second stimulation pulse based on the received stimulation signals and/or ECAP signals, and transmit instructions to second IMD 106B to cause the second IMD 106B to deliver the second stimulation pulse based on the adjusted stimulation parameters. In some examples, second IMD 106B may transmit the sensed stimulation signals and/or ECAP signals to a third IMD (not pictured). The third IMD may adjust stimulation parameters of first stimulation pulse and/or the second stimulation pulse based on the received stimulation signals and/or ECAP signals and instruct first IMD 106A and second IMD 106B to deliver the first stimulation pulse and the second stimulation pulse, respectively, in accordance with the adjusted stimulation parameters.

In some examples, external programmer 120 instead may perform one, several, or all of these functions. In such an example, each of IMDs 106 may transmit sensed signals (e.g., sensed stimulation signals, sensed ECAP signals) to external programmer 120 for analysis, and external programmer 120 may transmit instructions to each of IMDs 106 to adjust the one or more parameters defining the corresponding electrical stimulation therapies based on analysis of the sensed signals.

In some examples, stimulation generation circuitry of each of IMDs 106 may be configured to deliver at least one stimulation pulse at a same time as stimulation generation circuitry of another of IMDs 106 or at different times as the stimulation generation circuitry of another of IMDs 106. A timing difference between different stimulation pulses from the different IMDs 106 may be determined and/or adjusted by IMDs 106 and/or external programmer 120 based on sensed stimulation signals and/or ECAP signals from one or more of IMDs 106. IMDs 106 and/or external programmer 120 may adjust timing parameters of one or more stimulation pulses and/or the timing between different stimulation pulses, e.g., to optimize stimulation therapy efficacy, reduce unintended interference between the stimulation pulses, localize effects of stimulation therapy to a local region on patient 102, increase the area of effect of the stimulation therapy on patient 102, or the like. For example, IMD 106A may detect nerve propagation from pulses delivered by IMD 106B and then time the delivery of pulses from IMD 106A in order to avoid constructive signal propagation on the nerve that may cause the patient to perceive the nerve activity or perceive the nerve activity as uncomfortable.

In the example techniques described in this disclosure, the control stimulation parameters and the target stimulation signal value (e.g., a target range of characteristic values) of the stimulation signals may be initially set at the clinic but may be set and/or adjusted at home by patient 102. Once a target stimulation signal value (e.g., a target range of characteristic values) are set, the example techniques allow for automatic adjustment of parameters of the stimulation pulses in order to maintain consistent volume of neural activation and consistent perception of therapy for patient 102 when the electrode-to-neuron distance changes. The ability to change the stimulation parameter values may also allow the therapy to have long term efficacy, with the ability to keep the intensity of the stimulation (e.g., as indicated by the detected stimulation signals) consistent by comparing the measured characteristic values of the stimulation signals to the target range of characteristic values. IMDs 106 may perform these changes without intervention by a physician or patient 102.

In some examples, IMDs 106 are synchronized and/or controlled based on a bioelectric signal sensed by one or more of IMDs 106 and/or another sensor disposed on or within patient 102. The bioelectric signal may include, but are not limited to, local field potential (LFP) signals, electrocardiography (ECG) signals, electromyography (EMG) signals, or evoked signals such as ECAPs or evoked resonant neural activity (ERNA) signals. In some examples, one or more of IMDs 106 is configured to sense the bioelectrical signal from patient 102 and determine electrical stimulation parameters for one or more of IMDs 106 based on the sensed bioelectrical signal. The sensed bioelectrical signal and/or changes in the sensed bioelectrical signal may correspond to changes in the physiology of patient 102, changes in a disease state, effect of a pharmaceutical substance on patient 102, changes in brain activity of patient 102, or the like.

In some examples, one or more of IMDs 106 may be a medical device configured to deliver DBS to the brain of patient 102 and may include one or more electrodes configured to deliver electrical stimulation to and/or sense bioelectrical signals from the brain of patient 102. The bioelectrical signals may be generated autonomously by the brain of patient 102 (i.e., independent of any stimulation delivered by system 100) and may correspond to brain waves of patient 102 such as an LFP, a beta wave, an ERNA signal, or the like. The use of the sensed bioelectrical signal to adjust the stimulation parameters of IMDs 106 may allow system 100 to optimize the delivery of electrical stimulation by each of IMDs 106 to a desired stimulation therapy prior to the delivery of any stimulation signals by system 100 to patient 102.

Similar to how multiple devices may synchronize stimulation using ECAP signals described herein, multiple IMDs may coordinate stimulation delivery for DBS therapy. For example, one IMD may sense a brain wave, such as an LFP (inherent brain signal) or an ERNA (evoked signal), and adjust the timing of the delivery of one or more pulses in order for the pulses from each IMD to provide the desired effect. Without synchronization, multiple IMDs could deliver pulses that interfere with each other such that certain neurons do not receive the intended magnitude of the electric field. An example could be one pulse of an opposite polarity reducing the electric field of another pulse. This resulting decrease in electric field magnitude at certain neurons could render one or both pulses ineffective to change neuron function. With synchronization, one IMD may adjust the timing of pulses to be synchronized with another IMD, or both IMDs could time the pulses together, in order to avoid constructive or destructive interference from different pulses. This synchronization could be effective to maintain therapeutic efficacy from pulses of different IMDs when the pulses are not delivered at the same frequency (or multiples of each other). If frequencies are different, they may converge on each other. Effects of this frequency convergence may be reduced or eliminated by synchronizing the delivery of pulses at all times or when the pulses become too close in time to each other.

FIG. 2 is a block diagram illustrating an example configuration of components of IMD 200, in accordance with one or more techniques of this disclosure. IMD 200 may be an example of one of IMDs 106 of FIG. 1. In the example shown in FIG. 2, IMD 200 includes stimulation generation circuitry 202, switch circuitry 204, sensing circuitry 206, communication circuitry 208, processing circuitry 210, storage device 212, sensor(s) 222, and power source 224.

In the example shown in FIG. 2, storage device 212 stores therapy stimulation programs 214 and test stimulation programs 216 in separate memories within storage device 212 or separate areas within storage device 212. Each stored therapy stimulation program of therapy stimulation programs 214 defines values for a set of electrical stimulation parameters (e.g., a stimulation parameter set), such as a stimulation electrode combination, electrode polarity, current or voltage amplitude, pulse width, pulse rate, and pulse shape. In examples in which control pulses are provided to the patient without the need for informed pulses, a separate test stimulation program may not be needed. Instead, the test stimulation program for therapy that only includes control pulses may define the same control pulses as the corresponding therapy stimulation program for those control pulses.

Accordingly, in some examples, stimulation generation circuitry 202 generates electrical stimulation signals in accordance with the electrical stimulation parameters noted above. Other ranges of stimulation parameter values may also be useful and may depend on the target stimulation site within patient 102. While stimulation pulses are described, stimulation signals may be of any form, such as continuous-time signals (e.g., sine waves) or the like. Switch circuitry 204 may include one or more switch arrays, one or more multiplexers, one or more switches (e.g., a switch matrix or other collection of switches), or other electrical circuitry configured to direct stimulation signals from stimulation generation circuitry 202 to one or more of electrodes 220, 222, or directed sensed signals from one or more of electrodes 220, 222 to sensing circuitry 206. In other examples, stimulation generation circuitry 202 and/or sensing circuitry 206 may include sensing circuitry to direct signals to and/or from one or more of electrodes 220, 222, which may or may not also include switch circuitry 204.

Sensing circuitry 206 monitors signals from any combination of electrodes 220, 222. In some examples, sensing circuitry 206 includes one or more amplifiers, filters, and analog-to-digital converters. Sensing circuitry 206 may be used to sense physiological signals, such as ECAPs, local field potentials (LFPs), or other signals. Additionally, or alternatively, sensing circuitry 206 may sense one or more stimulation pulses delivered to patient 102 via electrodes 220, 222. In some examples, sensing circuitry 206 detects electrical signals, such as stimulation signals and/or ECAPs from a particular combination of electrodes 220, 222. In some cases, the particular combination of electrodes for sensing ECAPs includes different electrodes than a set of electrodes 220, 222 used to deliver stimulation pulses. Alternatively, in other cases, the particular combination of electrodes used for sensing ECAPs includes at least one of the same electrodes as a set of electrodes used to deliver stimulation pulses to patient 102. Sensing circuitry 206 may provide signals to an analog-to-digital converter, for conversion into a digital signal for processing, analysis, storage, or output by processing circuitry 210.

Communication circuitry 208 may be configured to support wireless communication between IMD 200 and an external programmer (not shown in FIG. 2) or another computing device (such as another IMD) under the control of processing circuitry 210. Processing circuitry 210 of IMD 200 may receive, as updates to programs, values for various stimulation parameters such as amplitude and electrode combination, from the external programmer via communication circuitry 208. Updates to the therapy stimulation programs 214 may be stored within storage device 212. Communication circuitry 208 in IMD 200, as well as communication circuits in other devices and systems described herein, such as the external programmer, may accomplish communication by radiofrequency (RF) communication techniques. In addition, communication circuitry 208 may communicate with an external medical device programmer (not shown in FIG. 2) via proximal inductive interaction of IMD 200 with the external programmer. The external programmer may be one example of external programmer 120 of FIG. 1. Accordingly, communication circuitry 208 may send information to the external programmer on a continuous basis, at periodic intervals, or upon request from IMD 106 or the external programmer.

Processing circuitry 210 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 controls stimulation generation circuitry 202 to generate stimulation signals according to therapy stimulation programs 214 stored in storage device 212 to apply stimulation parameter values specified by one or more of programs, such as amplitude, pulse width, pulse rate, and pulse shape of each of the stimulation signals.

In the example shown in FIG. 2, the set of electrodes 220 includes electrodes 220A, 220B, 220C, and 220D, and the set of electrodes 222 includes electrodes 222A, 222B, 222C, and 222D. In other examples, a single lead may include all eight electrodes 220 and 222 along a single axial length of the lead. Processing circuitry 210 also controls stimulation generation circuitry 202 to generate and apply the stimulation signals to selected combinations of electrodes 220, 222. In some examples, stimulation generation circuitry 202 includes a switch circuit (instead of, or in addition to, switch circuitry 204) that may couple stimulation signals to selected conductors within leads 230, which, in turn, deliver the stimulation signals across selected electrodes 220, 222. Such a switch circuit may be a switch array, switch matrix, multiplexer, or any other type of switching circuit configured to selectively couple stimulation energy to selected electrodes 220, 222 and to selectively sense bioelectrical neural signals of a spinal cord of the patient (not shown in FIG. 2) with selected electrodes 220, 222.

In other examples, however, stimulation generation circuitry 202 does not include a switch circuit and switch circuitry 204 does not interface between stimulation generation circuitry 202 and electrodes 220, 222. In these examples, stimulation generation circuitry 202 includes a plurality of pairs of voltage sources, current sources, voltage sinks, or current sinks connected to each of electrodes 220, 222 such that each pair of electrodes has a unique signal circuit. In other words, in these examples, each of electrodes 220, 222 is independently controlled via its own signal circuit (e.g., via a combination of a regulated voltage source and sink or regulated current source and sink), as opposed to switching signals between electrodes 220, 222.

Electrodes 220, 222 on respective leads 108 may be constructed of a variety of different designs. For example, one or both of leads 108 may include one or more electrodes at each longitudinal location along the length of the lead, such as one electrode at different perimeter locations around the perimeter of the lead at each of the locations A, B, C, and D. In one example, the electrodes may be electrically coupled to stimulation generation circuitry 202, e.g., via switch circuitry 204 and/or switching circuitry of the stimulation generation circuitry 202, via respective wires that are straight or coiled within the housing of the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 108. These and other constructions may be used to create a lead with a complex electrode geometry.

Although sensing circuitry 206 is incorporated into a common housing with stimulation generation circuitry 202 and processing circuitry 210 in FIG. 2, in other examples, sensing circuitry 206 may be in a separate housing from IMD 200 and may communicate with processing circuitry 210 via wired or wireless communication techniques. In some examples, sensing circuitry 206 may be disposed in a separate IMD and may communicate with IMD 200 via wired or wireless communication techniques.

In some examples, one or more of electrodes 220 and 222 are suitable for sensing one or more ECAPs. For instance, electrodes 220 and 222 may sense the voltage amplitude of a portion of the ECAP signals, where the sensed voltage amplitude is a characteristic of the ECAP signal.

In some examples, one or more of electrodes 220 and 222 are suitable for sensing stimulation signals. For instance, electrodes 220 and 222 may sense the voltage amplitude of a portion of the stimulation signals, where the sensed voltage amplitude is a characteristic of the stimulation signals. In some examples, one or more of electrodes 220 and 222 may sense a stimulation signal in response to one or more of electrodes 220 and 222 delivering a stimulation pulse to target tissue of patient 102. In some examples, the one or more of electrodes 220 and 222 which sense the stimulation signal are not the same as the one or more of electrodes 220 and 222 which deliver the stimulation pulse.

Storage device 212 may be configured to store information within IMD 200 during operation. Storage device 212 may include a computer-readable storage medium or computer-readable storage device. In some examples, storage device 212 includes one or more of a short-term memory or a long-term memory. Storage device 212 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 212 is used to store data indicative of instructions for execution by processing circuitry 210. As discussed above, storage device 212 is configured to store therapy stimulation programs 214.

In some examples, stimulation generation circuitry 202 may be configured to deliver electrical stimulation therapy to patient 102. In some examples, the electrical stimulation therapy may include a plurality of informed pulses. Additionally, stimulation generation circuitry 202 may be configured to deliver a plurality of control pulses, where the plurality of control pulses is interleaved with at least some informed pulses of the plurality of informed pulses. Stimulation generation circuitry 202 may deliver the plurality of informed pulses and the plurality of control pulses to target tissue (e.g., spinal cord 104) of patient 102 via electrodes 220, 222 of leads 108. By delivering such informed pulses and control pulses, stimulation generation circuitry 202 may cause IMD 200 to sense stimulation signals that are indicative of the delivered pulses Additionally, or alternatively, stimulation generation circuitry 202 may deliver control pulses that evoke detectable responsive ECAPs in the target tissue, the responsive ECAPs propagating through the target tissue before arriving back at electrodes 220, 222. Stimulation signals or ECAPs caused by or elicited by informed pulses may not be detectable by IMD 200. In some examples, a different combination of electrodes 220, 222 may sense responsive ECAPs and/or responsive stimulation signals than a combination of electrodes 220, 222 that delivers informed pulses and a combination of electrodes 220, 222 that delivers control pulses. Sensing circuitry 206 may be configured to detect the responsive ECAPs and/or the responsive stimulation signals via electrodes 220, 222 and leads 108. In other examples, stimulation generation circuitry 202 may be configured to deliver a plurality of control pulses, without any informed pulses, when control pulses also provide or contribute to a therapeutic effect for the patient.

In some examples, stimulation generation circuitry 202 may deliver multimodal stimulation (e.g., differential targeted multiplexed stimulation). Multimodal stimulation includes different stimulation pulses (e.g., a prime stimulation pulse and a base stimulation pulse) defined by different stimulation parameters such as different frequencies and different electrode combinations. Since different types of cells, such as glial cells and neurons, respond differently to electrical fields, it is then possible to differentially modulate the response of these cell populations with distinctly different electrical parameters. For example, the prime stimulation pulses can be delivered to affect glial cells (e.g., in spinal cord 104) and the base stimulation pulses can be delivered to affect neurons. Generally, the prime stimulation pulses is delivered at a higher pulse frequency than the base stimulation pulses, as described in greater detail herein.

Processing circuitry 210 may, in some cases, direct sensing circuitry 206 to continuously monitor for ECAPs and stimulation signals. In other cases, processing circuitry 210 may direct sensing circuitry 206 to monitor for ECAPs and stimulation signals based on signals from sensor(s) 222. Activating and deactivating sensing circuitry 206 may, in some examples, extend a battery life of power source 224.

Processing circuitry 210 may determine timing parameters of stimulation pulses delivered by IMD 200. The timing parameters may be applied by IMD 200 relative to one or more other stimulation pulses delivered by other IMDs (e.g., another IMD 200). Processing circuitry 210 may determine the timing parameters of stimulation pulses based on user input (e.g., from external device 120). The timing parameters of stimulation pulses may be based on a predetermined interaction between different stimulation pulses for a particular therapy stimulation program 214. For example, a therapy stimulation program 214 may cause the stimulation pulses to propagate, amplify, diminish, or cancel out one or more of the stimulation pulses, e.g., by constructive or destructive interference. In some examples, therapy stimulation programs 214 may cause the stimulation pulses to propagate, amplify, diminish, or cancel out one or more of the stimulation pulses by synchronizing the delivery of the stimulation pulses, adjusting the timing of the delivery of the stimulation pulses such that a target tissue of patient 102 (e.g., a peripheral nerve of patient 102) receives multiple stimulation pulses simultaneously, or by adjusting the timing of the delivery of the stimulation pulses such that the target tissue of patient 102 receives each stimulation pulse after a predetermined period of time following a previously received stimulation pulse.

Although the timing parameters may be directed to coordinating the timing of the delivered pulses to other sensed pulses, the timing parameters may instead control the delivery of stimulation pulses to manage the interaction of the delivered stimulation pulses to the action potentials propagating along one or more nerve fibers. For example, processing circuitry 210 may time the delivery of stimulation to increase the propagating action potentials (e.g., construction interference) or reduce the propagation of action potentials (e.g., destructive interference). In this manner, processing circuitry 210 may be configured to directly modulate electrical signals from other implanted devices and/or modulate propagating signals in the nerves as a result of stimulation from another device.

Processing circuitry 210 may sense, via sensing circuitry 206, stimulation signals and/or ECAP signals corresponding to stimulation pulses from other IMDs 200. Processing circuitry 210 may determine a timing of the stimulation pulses from other IMDs 200 based on the sensed stimulation signals and/or ECAP signals and cause stimulation generation circuitry 202 to deliver stimulation pulses to deliver stimulation pulses based on the determined timing parameters of the stimulation pulse and relative to the other delivered stimulation pulses from other IMDs 200. For example, the timing difference between delivered stimulation and the resulting ECAP signal detected by another IMD may indicate how long it takes for action potentials to propagate from the electrodes of one IMD to the electrodes of another IMD at the different tissue location. This timing may then be used to coordinate timing of stimulation from each IMD, or at least the IMD delivering stimulation to nerves through which the ECAP propagates, in order to achieve the desired action potential propagation. The IMD that sensed the ECAP may determine the specific time to deliver one or more stimulation pulses that may enhance or reduce the action potential propagating through that tissue.

Processing circuitry 210 may determine, based on the sensed stimulation signals and/or ECAP signals, a first time when a target tissue of patient 102 receives a first stimulation signal from another IMD 200. Based on the determined first time and predetermined timing parameters of a selected therapy stimulation program 214, processing circuitry 210 may cause stimulation generation circuitry 202 to deliver a second stimulation pulse to the target tissue of patient 102 at a second time, e.g., such that a time difference between the first time and the second time is consistent with the predetermined timing parameters.

In some examples, a selected therapy stimulation program 214 may include predetermined timing parameters corresponding to synchronization of the delivery of stimulation pulses from multiple IMDs 200 and/or synchronization of the reception of stimulation pulses from multiple IMDs 200 by a specific target tissue of patient 102 (e.g., by a specific peripheral nerve of patient 102). In such examples, processing circuitry 210 may adjust the second time based on the sensed stimulation signals and/or ECAP signals to cause stimulation generation circuitry 202 to deliver the second stimulation pulses consistent with the selected therapy stimulation program 214.

In some examples, a selected therapy stimulation program 214 may include predetermined timing parameters corresponding to an offset of the delivery of the first stimulation pulse and the second stimulation pulse by a predetermined period of time and/or offset of the reception, by a specific target tissue of patient 102 (e.g., by a specific peripheral nerve of patient 102), of the first stimulation pulse and the second stimulation pulse by the predetermined period of time. In such examples, processing circuitry 210 may adjust the second time based on the sensed stimulation signals and/or ECAP signals to cause stimulation generation circuitry 202 to deliver the second stimulation pulse consistent with the selected therapy stimulation program 214 to cause the offset between delivery and/or reception of the first stimulation pulse and the second stimulation pulse by the predetermined period of time.

Processing circuitry 210 may continuously and/or periodically monitor the sensed stimulation signals and/or evoked signals, such as ECAP signals. Processing circuitry 210 may determine deviations from predetermined timing parameters of one or more of the stimulation pulses and/or of the selected therapy stimulation program 214 (e.g., due to changes in physiology of patient 102, due to progression of a medical condition, due to the presence of new medical conditions) and may adjust stimulation parameters (e.g., timing parameters) of the stimulation pulses to maintain the predetermined timing. For example, processing circuitry 210 may increase a pulse width and/or a pulse amplitude of one or more of the stimulation pulses to account for changes in the medical condition of patient 102. In some examples, processing circuitry 210 may predict, based on the determined deviations from the predetermined timing parameters, additional deviation in the future. Processing circuitry 210 may adjust the stimulation parameters of the stimulation pulses to account for the predicted deviation.

Power source 224 is configured to deliver operating power to the components of IMD 200. Power source 224 may include a battery and a power generation circuit to produce the operating power. In some examples, the battery is rechargeable to allow extended operation. In some examples, recharging is accomplished through proximal inductive interaction between an external charger and an inductive charging coil within IMD 200. Power source 224 may include any one or more of a plurality of different battery types, such as nickel cadmium batteries and lithium ion batteries.

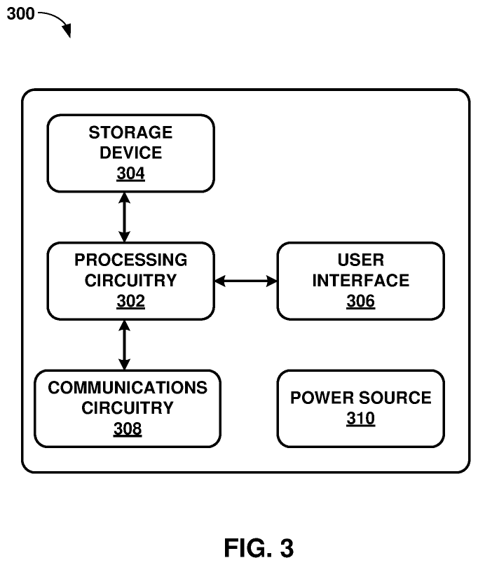

FIG. 3 is a block diagram illustrating an example configuration of components of external programmer 300, in accordance with one or more techniques of this disclosure. External programmer 300 may be an example of external programmer 120 of FIG. 1. Although external programmer 300 may generally be described as a hand-held device, external programmer 300 may be a larger portable device or a more stationary device. In addition, in other examples, external programmer 300 may be included as part of an external charging device or include the functionality of an external charging device. As illustrated in FIG. 3, external programmer 300 may include processing circuitry 302, storage device 304, user interface 306, communications circuitry 358, and power source 310. Storage device 304 may store instructions that, when executed by processing circuitry 302, cause processing circuitry 302 and external programmer 300 to provide the functionality ascribed to external programmer 300 throughout this disclosure. Each of these components, circuitry, or modules, may include electrical circuitry that is configured to perform some, or all of the functionality described herein. For example, processing circuitry 302 may include processing circuitry configured to perform the processes discussed with respect to processing circuitry 302.

In general, external programmer 300 includes any suitable arrangement of hardware, alone or in combination with software and/or firmware, to perform the techniques attributed to external programmer 300, and processing circuitry 302, user interface 306, and communications circuitry 308 of external programmer 300. In various examples, external programmer 300 may include one or more processors, such as one or more microprocessors, DSPs, ASICs, FPGAs, or any other equivalent integrated or discrete logic circuitry, as well as any combinations of such components. External programmer 300 also, in various examples, may include a storage device 304, such as RAM, ROM, PROM, EPROM, EEPROM, flash memory, a hard disk, a CD-ROM, including executable instructions for causing the one or more processors to perform the actions attributed to them. Moreover, although processing circuitry 302 and communications circuitry 308 are described as separate modules, in some examples, processing circuitry 302 and communications circuitry 308 are functionally integrated. In some examples, processing circuitry 302 and communications circuitry 308 correspond to individual hardware units, such as ASICs, DSPs, FPGAs, or other hardware units.

Storage device 304 (e.g., a storage device) may store instructions that, when executed by processing circuitry 302, cause processing circuitry 302 and external programmer 300 to provide the functionality ascribed to external programmer 300 throughout this disclosure. For example, storage device 304 may include instructions that cause processing circuitry 302 to instruct IMDs 106 to deliver stimulation pulses to patient 102 in accordance with one or more therapy stimulation programs 214 (not pictured) stored in one or more of IMDs 106 or external programmer 300. In some examples, storage device 304 may include instructions that cause processing circuitry 302 to request sensed stimulation signals and/or ECAP signals from one or more of IMDs 106 and adjust timing of one or more stimulation pulses configured to be delivered by IMDs 106 based on the sensed stimulation signals and/or ECAP signals. The instructions may cause processing circuitry 302 to adjust the timing parameters of one or more of the stimulation pulses based on the sensed stimulation signals and/or ECAP signals, e.g., in accordance with the stimulation parameters of therapy stimulation program 214.

Storage device 304 may include a plurality of therapy stimulations programs, where each program includes a parameter set that defines stimulation pulses, such as prime stimulation pulses and/or base stimulation pulses. Storage device 304 may also store data received from a medical device (e.g., IMD 106). The data may include, but is not limited to, sensed stimulation signals, sensed ECAP signals, and stimulation parameters (e.g., timing parameters) for a stimulation pulse configured to be delivered by the medical device. For example, storage device 304 may store stimulation signals and/or ECAP related data recorded at a sensing module of the medical device, and storage device 304 may also store data from one or more sensors of the medical device.

User interface 306 may include a button or keypad, lights, a speaker for voice commands, a display, such as a liquid crystal (LCD), light-emitting diode (LED), or organic light-emitting diode (OLED). In some examples the display includes a touch screen. User interface 306 may be configured to display any information related to the delivery of electrical stimulation, identified patient behaviors, sensed patient parameter values, patient behavior criteria, or any other such information. User interface 306 may also receive user input via user interface 306. The input may be, for example, in the form of pressing a button on a keypad or selecting an icon from a touch screen. The input may request a particular therapy stimulation program 214 from a plurality of therapy stimulation programs 214. In some examples, the input may request a particular type of medical therapy (e.g., pain relief with paresthesia, pain relief without paresthesia, localized pain relief, etc.). Each particular type of medical therapy may be delivered by two or more separate stimulation pulses delivered by two or more IMDs 106 at particular times (e.g., with respect to another stimulation pulse).

Communications circuitry 308 may support wireless communication between the medical device and external programmer 300 under the control of processing circuitry 302. Communications circuitry 308 may also be configured to communicate with another computing device via wireless communication techniques, or direct communication through a wired connection. In some examples, communications circuitry 308 provides wireless communication via an RF or proximal inductive medium. The another computing device may include a medical device (e.g., a wearable medical device, an IMD) configured to communicate with IMDs 106 and external programmer 300. In such examples, IMDs 106 may communicate with external programmer 300 via the medical device and may not directly communicate with external programmer 300. In some examples, communications circuitry 308 includes an antenna, which may take on a variety of forms, such as an internal or external antenna.

Examples of local wireless communication techniques that may be employed to facilitate communication between external programmer 300 and IMD 106 include RF communication according to the 802.11 or Bluetooth® specification sets or other standard or proprietary telemetry protocols. In this manner, other external devices may be capable of communicating with external programmer 300 without needing to establish a secure wireless connection. As described herein, communications circuitry 308 may be configured to transmit a spatial electrode movement pattern or other stimulation parameter values to one or more of IMDs 106 for delivery of electrical stimulation therapy.

In some examples, stimulation parameters (e.g., timing parameters) of therapy stimulation programs are transmitted to one or more IMDs 106 for delivery to a patient (e.g., patient 102 of FIG. 1). In some examples, programmer 300 may coordinate the delivery of stimulation by instructing stimulation delivery from each of IMDs 106 at specific times. Programmer 300 may send clock signals that enable the IMDs to maintain synchronized clocks for enabling the coordination of stimulation delivery. In other examples, the therapy may include medication, activities, or other instructions that patient 102 may perform themselves or a caregiver perform for patient 102. In some examples, external programmer 300 provides visual, audible, and/or tactile notifications that indicate there are new instructions. External programmer 300 requires receiving user input acknowledging that the instructions have been completed in some examples.

According to the techniques of the disclosure, user interface 306 of external programmer 300 receives an indication from a clinician instructing a processor of the medical device to update one or more therapy stimulation programs or to update one or more test stimulation programs. Updating therapy stimulation programs and test stimulation programs may include changing one or more parameters (e.g., timing parameters) of the stimulation pulses delivered by IMDs according to the programs, such as amplitude, pulse width, frequency, timing, pulse shape, and/or type of pulse (e.g., informed pulses, control pulses, prime pulses, base pulses). User interface 306 may also receive instructions from the clinician commanding any electrical stimulation, including control pulses and/or informed pulses to commence or to cease.

Power source 310 is configured to deliver operating power to the components of external programmer 300. Power source 310 may include a battery and a power generation circuit to produce the operating power. In some examples, the battery is rechargeable to allow extended operation. Recharging may be accomplished by electrically coupling power source 310 to a cradle or plug that is connected to an alternating current (AC) outlet. In addition, recharging may be accomplished through proximal inductive interaction between an external charger and an inductive charging coil within external programmer 300. In other examples, traditional batteries (e.g., nickel cadmium or lithium ion batteries) may be used. In addition, external programmer 300 may be directly coupled to an alternating current outlet to operate.

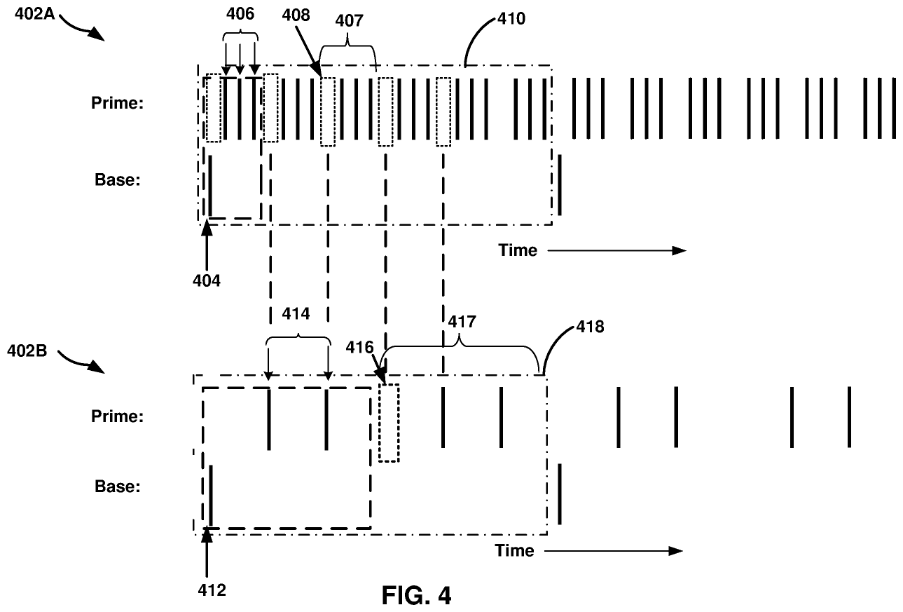

FIG. 4 is a graph 400 illustrating example timing diagrams for the delivery of stimulation pulses by the plurality of IMDs 106 of FIG. 1, in accordance with one or more techniques of this disclosure. As illustrated in FIG. 4, graph 400 includes a first timing diagram 402A and a second timing diagram 402B (collectively referred to as “timing diagrams 402”). In some examples, graph 400 may include three or more timing diagrams. Timing diagrams 402 may be an example of multimodal stimulation, but the same coordination of signals delivered by different IMDs could be used in other example single therapies or separate therapies delivered over the same time period.

Each of timing diagrams 402 illustrate delivery of stimulation pulses by an IMD (e.g., one of IMDs 106) over time. For example, as illustrated in FIG. 4, first timing diagram 402A illustrates stimulation pulses delivered by first IMD 106A and second timing diagram 402B illustrates stimulation pulses delivered by second IMD 106A. Each of timing diagrams 402 may illustrate different stimulation pulses delivered by the corresponding IMD 106 to patient 102 include, but are not limited to, prime stimulation pulses and base stimulation pulses of differential targeted multimodal stimulation (e.g., as illustrated in FIG. 4), control pulses and informed pulses, or sub-action-potential-threshold pulses (also referred to as “subthreshold pulses,” “subthreshold stimulation pulses) and supra-threshold-action-potential pulses (also referred to as “suprathreshold pulses,” “suprathreshold stimulation pulses”). Subthreshold pulses may include stimulation pulses that have an intensity that does not elicit an action potential threshold and suprathreshold pulses may include stimulation pulses that have an intensity greater than the action potential threshold which causes an action potential. In other examples, the threshold may refer to a perception threshold, motor threshold, or other threshold. The action potential threshold represents a minimum threshold voltage required to cause a nerve of patient 102 to output an action potential (e.g., an ECAP).

First timing diagram 402A illustrates to the timing of the delivery of stimulation pulses to target tissue of patient 102 by a first IMD (e.g., first IMD 106A). First timing diagram 402A illustrates the delivery of base pulses 404 and prime pulses 406 in cycles 410. In some examples, the stimulation pulses illustrated in first timing diagram 402A are delivered by a single IMD. For example, first IMD 106A may deliver base pulses 404 to a first target tissue in patient 102 (e.g., a peripheral nerve of patient 102) via a first lead (e.g., lead 108A) and deliver prime pulses 406 to a second target tissue in patient 102 (e.g., glial cells in spinal cord 104) via a second lead (e.g., lead 108B). In some examples, the stimulation pulses illustrated in first timing diagram 402A are delivered by two or more IMDs. For example, a first IMD may deliver base pulses 404 to the first target tissue and a second IMD may deliver prime pulses 406 to the second target tissue.

As illustrated in first timing diagram 402A, base pulses 404 may be delivered at a lower frequency than prime pulses 406, e.g., to allow one or more of IMDs 106 to sense ECAP signals from a pulse of base pulses 404. In such examples, base pulses 404 may be control pulses and prime pulses 406 may be informed pulses. Each of base pulses 404 and prime pulses 406 may be defined by a plurality of parameters including, but are not limited to, pulse amplitude (current and/or voltage), pulse frequency, pulse width, pulse shape, and/or electrode combination).

Each cycle 410 of stimulation pulses may include one or more base pulses 404 and one or more prime pulses 406. Each cycle 410 may be defined by the delivery of one base pulse 404. In other examples, each cycle 410 may be defined in another manner, e.g., by the delivery of a specific number of prime pulses 406. Each cycle 410 includes a plurality of segments 407, each segment 407 defining a plurality of slots 408. Within each segment 407, slots 408 may be separated by a period of time. The period of time may be based on the pulse frequency and/or pulse width of base pulse 404 and/or prime pulse 406. IMD 106A may deliver a base pulse 404 and/or prime pulses 406 in each of the slots 408. In other examples, prime pulses 406 may be delivered at any single or varying frequency as desired for therapy.

As illustrated in FIG. 4, IMD 106A by deliver base pulse 404 in a first slot 408 of a first segment 407 of each cycle 410. In other examples, IMD 106A may deliver base pulses 404 in another slot 408 and/or in another segment 407 of each cycle 410. Delivery of a single base pulse 404 for each cycle 410 may allow IMD 106A sufficient time to detect and analyze the ECAP signals resulting from base pulse 404. IMD 106A may deliver prime pulses 406 in the remaining slots 408 of the first segment 407 of each cycle 410. In some examples, as illustrated in FIG. 4, IMD 106A may not deliver any stimulation pulses in the first slots 408 of subsequent segments 407 of each cycle 410, e.g., to maintain a pulse frequency of prime pulses 406 throughout each cycle 410. In some examples, the stimulation parameters of base pulses 404 and/or prime pulses 406 may be constant across multiple cycles 410. In some examples, the stimulation parameters of base pulses 404 and/or prime pulses 406 may vary between different cycles 410. When pulses are not delivered by IMD 106A, IMD 106A may sense for other stimulation pulses delivered by another IMD and/or action potentials propagating, such as ECAPs. A time period such as this may enable the sensing of a pulse or resulting ECAP from the prime or base pulses in timing diagram 402B.

Second timing diagram 402B may illustrate base pulses 412 and prime pulses 414 delivered by a second IMD (e.g., IMD 106B). Second timing diagram 402B may define a plurality of cycles 418, each cycle 418 defining a plurality of segments 417 and each segment 417 defining a plurality of slots 416. As illustrated, cycle 418 may be a same length as cycle 410, e.g., to allow for repeatable and consistent delivery of stimulation pulses by IMDs 106.

As illustrated in second timing diagram 402B, IMD 106B may deliver base pulses 412 and prime pulses 414 with different stimulation parameters (e.g., with different pulse frequencies, pulse widths, or the like) than base pulses 404 and prime pulses 406 delivered by IMD 106A. Cycle may include a different number of segments 417 than a number of segments 407 in cycle 410 and each of segments 417 may have a different number of slots 416 than a number of slots 408 in segment 407. Within each of segments 417, slots 416 may be separated by different period of time than slots 408 within segments 417. The differences outlined above may be due to the difference in stimulation parameters between base pulses 404 and base pulses 412 and/or between prime pulses 406 and prime pulses 414. In some examples, the stimulation parameters for base pulses 404 and base pulses 412 may be the same and the stimulation parameters for prime pulses 406 and prime pulses 414 may be different.

In some examples, the stimulation parameters for the stimulation pulses in timing diagrams 402 may be determined such that the delivery of stimulation pulses by one of IMDs 106 (e.g., IMD 106B) may coincide with slots in the stimulation cycle (e.g., slots 408) and/or stimulation pulses (e.g., prime pulses 406) delivered by another of IMDs 106 (e.g., IMD 106A). For example, IMD 106B may deliver a prime pulse 414 and IMD 106A may deliver a prime pulse 406 at a same time. In some examples, as illustrated in FIG. 4, IMD 106B may deliver prime pulse 414 to patient 102 when IMD 106A does not deliver prime pulse 406. Alternating delivery of prime pulses (e.g., prime pulses 406 and 414) between two or more IMDs 106 may reduce power consumption of each of IMDs 106 and increase battery life of IMDs 106.

In some examples, IMD 106B and IMD 106A may deliver prime pulses at a same time to perform one or more functions. In some examples, prime pulses 414 may amplify and/or propagate prime pulses 406, e.g., due to constructive interference of the stimulation pulses themselves (e.g., modulating the electrical field experienced from both pulses) or for the action potentials propagating within the tissue. In such examples, prime pulses 414 may extend the transmission and effects of prime pulses 406 across a larger volume of target tissue of patient 102. In some examples, prime pulses 414 may diminish and/or cancel out prime pulses 406, e.g., due to destructive interference. diminishing and/or cancelling out prime pulses 406 may reduce patient discomfort. For example, diminishing and/or cancelling out of prime pulses 406 may prevent and/or reduce propagation of paresthesia outside a localized region on patient 102. In some examples, prime pulses 414 may diminish and/or cancel out ECAP signals resulting from prime pulses 406, e.g., thereby preventing and/or reducing propagation of paresthesia outside the localized region.

In some examples, patient 102 may require the delivery of subthreshold pulses, e.g., to reduce or eliminate pain. In such examples, IMDs 106 may detect stimulation signals in the nerves of patient 102 to determine and/or adjust the timing of different stimulation pulses, in accordance with the example methods and techniques described herein. In some examples, IMDs 106 may deliver suprathreshold pulses between adjacent subthreshold pulses, at a certain frequency within the stimulation pulses (e.g., one suprathreshold stimulation pulse every minute), or in response to a request by another of IMDs 106 or external programmer 120. The suprathreshold pulse may cause the nerves to generate ECAP signals and the ECAP signals may be detected by other IMDs 106 to determine the timing of the corresponding stimulation pulse of the ECAP signal.

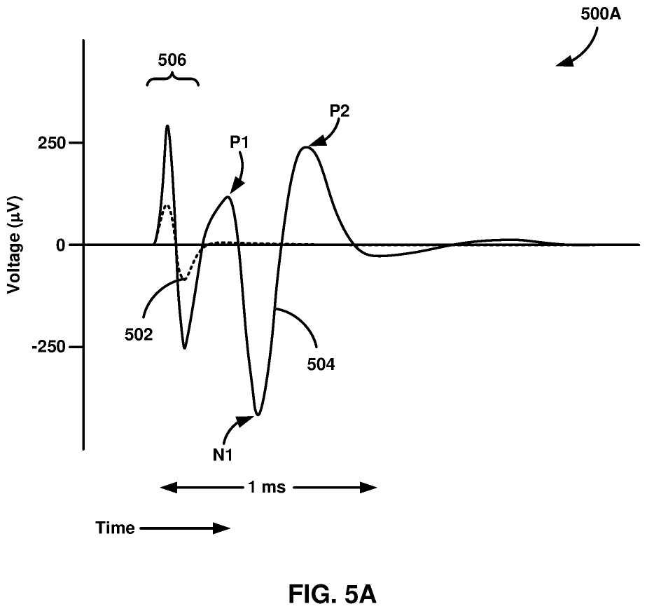

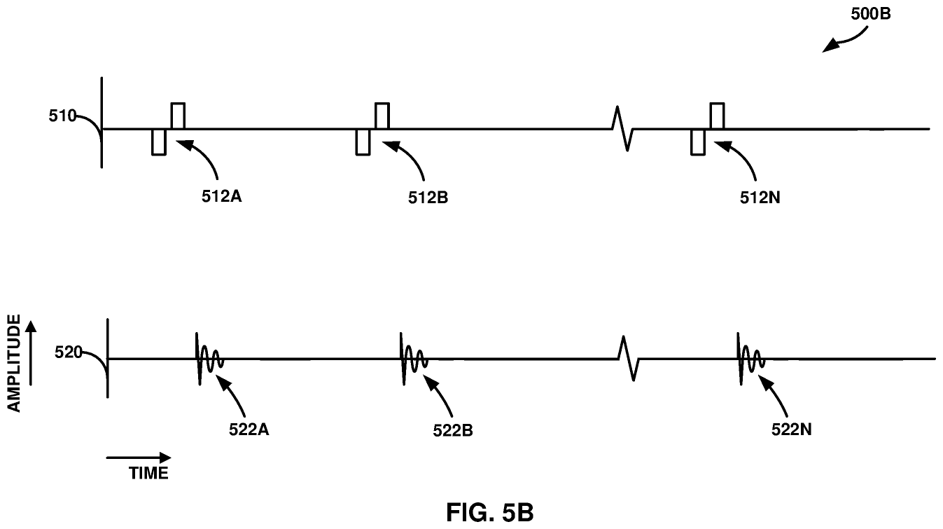

FIG. 5A is a graph 500A of example evoked compound action potentials (ECAPs) sensed for respective stimulation pulses, in accordance with one or more techniques of this disclosure. As shown in FIG. 4, graph 500A shows example ECAP signal 502 (dotted line) and ECAP signal 504 (solid line). In some examples, each of ECAP signals 502 and 504 are sensed from stimulation pulses (e.g., a control pulse, a base pulse) that were delivered from a guarded cathode, where the stimulation pulses are bi-phasic pulses including an interphase interval between each positive and negative phase of the pulse. In some such examples, the guarded cathode includes stimulation electrodes located at the end of an 8-electrode lead (e.g., leads 108 of FIG. 1) while two sensing electrodes are provided at the other end of the 8-electrode lead. ECAP signal 502 illustrates the voltage amplitude sensed as a result from a sub-detection threshold stimulation pulse, or a stimulation pulse which results in no detectable ECAP. It is noted that monophasic, tri-phasic, or pulses with another quantity of phases may be in other examples.

Peaks 506 of ECAP signal 502 are detected and represent stimulation signals of the delivered stimulation pulse. However, no propagating signal is detected after the stimulation signal in ECAP signal 502 because the stimulation pulse had an intensity (e.g., an amplitude and/or pulse width) that was “subthreshold” or below a detection threshold (e.g., a sub-detection threshold) and/or below a propagation threshold (e.g., a sub-propagation threshold).

In contrast to ECAP signal 502, ECAP signal 504 represents the voltage amplitude detected from a supra-detection stimulation threshold stimulation pulse. Peaks 506 of ECAP signal 504 are detected and represent stimulation signals of the delivered stimulation pulse. After peaks 506, ECAP signal 504 also includes peaks P1, N1, and P2, which are three typical peaks representative of propagating action potentials from an ECAP. The example duration of the stimulation signal and peaks P1, N1, and P2 is approximately 1 millisecond (ms).

When detecting the ECAP of ECAP signal 504, different characteristics may be identified. For example, the characteristic of the ECAP may be the amplitude between N1 and P2. This N1−P2 amplitude may be easily detectable even if the stimulation signal impinges on P1, a relatively large signal, and the N1−P2 amplitude may be minimally affected by electronic drift in the signal. In other examples, the characteristic of the ECAP used to control subsequent stimulation pulses (e.g., control pulses and/or informed pulses) may be an amplitude of P1, N1, or P2 with respect to neutral or zero voltage. In some examples, the characteristic of the ECAP used to control subsequent stimulation pulses is a sum of two or more of peaks P1, N1, or P2. In other examples, the characteristic of ECAP signal 504 may be the area under one or more of peaks P1, N1, and/or P2. In other examples, the characteristic of the ECAP may be a ratio of one of peaks P1, N1, or P2 to another one of the peaks. In some examples, the characteristic of the ECAP is a slope between two points in the ECAP signal, such as the slope between N1 and P2. In other examples, the characteristic of the ECAP may be the time between two points of the ECAP, such as the time between N1 and P2.

The time between when the stimulation pulse is delivered and a point in the ECAP signal may be referred to as a latency of the ECAP and may indicate the types of fibers being captured by the stimulation pulse (e.g., a control pulse). ECAP signals with lower latency (i.e., smaller latency values) indicate a higher percentage of nerve fibers that have faster propagation of signals, whereas ECAP signals with higher latency (i.e., larger latency values) indicate a higher percentage of nerve fibers that have slower propagation of signals. Latency may also refer to the time between an electrical feature is detected at one electrode and then detected again at a different electrode. This time, or latency, is inversely proportional to the conduction velocity of the nerve fibers. Other characteristics of the ECAP signal may be used in other examples.

The amplitude of the ECAP signal increases with increased amplitude of the stimulation pulse, as long as the pulse amplitude is greater than threshold such that nerves depolarize and propagate the signal. The target ECAP characteristic (e.g., the target ECAP signal amplitude) may be determined from the ECAP signal detected from a stimulation pulse (or a control pulse) when informed pulses are determined to deliver effective therapy to patient 102. The ECAP signal thus is representative of the distance between the stimulation electrodes and the nerves appropriate for the stimulation parameter values of the informed pulses delivered at that time. Therefore, one or more of IMDs 106 may attempt to use detected changes to the measured ECAP characteristic value to change therapy pulse parameter values and maintain the target ECAP characteristic value during therapy pulse delivery. In some examples, one or more of IMDs 106 (e.g., IMD 106B) may receive from an IMD 106 (e.g., IMD 106A) of timing of the delivered stimulation pulses. IMD 106B may then determine a nerve conduction velocity based on an amount of time between IMD 106A delivering the stimulation pulses and IMD 106B sensing the ECAP signals from the stimulation pulses. Based on the determined nerve conduction velocity, IMD 106B and/or external programmer 120 may adjust the timing of stimulation pulses configured to be delivered by IMD 106B, e.g., to coordinate the stimulation pulses delivered by IMD 106A and IMD 106B in accordance with a predetermined therapy stimulation program (e.g., therapy stimulation program 214).

FIG. 5B is a timing diagram 500B illustrating an example of electrical stimulation pulses, respective stimulation signals, and respective sensed ECAPs, in accordance with one or more techniques of this disclosure. For convenience, FIG. 5B is described with reference to IMD 200 of FIG. 2. As illustrated, timing diagram 500B includes first channel 510, a plurality of stimulation pulses 512A-512N (collectively “stimulation pulses 512”), second channel 520, and a plurality of respective ECAPs 522A-522N (collectively “ECAPs 522”). In some examples, stimulation pulses 512 may represent control pulses which are configured to elicit ECAPs 522 that are detectible by IMD 200, but this is not required. In some examples, stimulation pulses 512 may represent base pulses (e.g., base pulses 404, base pulses 512) Stimulation pulses 512 may represent any type of pulse that is deliverable by IMD 200. In the example of FIG. 5B, IMD 200 can deliver therapy with control pulses instead of, or without, informed pulses.

First channel 510 is a time/voltage (and/or current) graph indicating the voltage (or current) of at least one electrode of electrodes 220, 222. In one example, the stimulation electrodes of first channel 510 may be located on the opposite side of the lead as the sensing electrodes of second channel 520. Stimulation pulses 512 may be electrical pulses delivered to the spinal cord of the patient by at least one of electrodes 220, 222, and may be shown with a negative phase and a positive phase separated by an interphase interval. For example, a stimulation pulse 512 may have a negative voltage for the same amount of time and amplitude that it has a positive voltage. It is noted that the negative voltage phase may be before or after the positive voltage phase. Stimulation pulses 512 may be delivered according to therapy stimulation programs 214 stored in storage device 212 of IMD 200, and therapy stimulation programs 214 may be updated according to user input via an external programmer and/or may be updated according to a signal from sensor(s) 222. The signal may include a stimulation signal and/or ECAP signal corresponding to a stimulation pulse delivered by another IMD (e.g., another of IMDs 106). Each of stimulation pulses 512 may have a pulse width of less than approximately 300 microseconds (e.g., the total time of the positive phase, the negative phase, and the interphase interval is less than 300 microseconds). In some examples, each of stimulation pulses 512 may have a pulse width of approximately 100 □s for each phase of the bi-phasic pulse. As illustrated in FIG. 5B, stimulation pulses 512 may be delivered via channel 510. Delivery of stimulation pulses 512 may be delivered by leads 108 in a guarded cathode electrode combination. For example, if leads 108 are linear 8-electrode leads, a guarded cathode combination is a central cathodic electrode with anodic electrodes immediately adjacent to the cathodic electrode.

Second channel 520 is a time/voltage (and/or current) graph indicating the voltage (or current) of at least one electrode of electrodes 220, 222. In one example, the electrodes of second channel 520 may be located on the opposite side of the lead as the electrodes of first channel 510. ECAPs 508 may be sensed at electrodes 220, 222 from the spinal cord of the patient in response to stimulation pulses 512. ECAPs 522 are electrical signals which may propagate along a nerve away from the origination of stimulation pulses 512. In one example, ECAPs 522 are sensed by different electrodes than the electrodes used to deliver stimulation pulses 512. As illustrated in FIG. 5B, ECAPs 508 may be recorded on second channel 520. The ECAPs described in FIGS. 5A and 5B can be used to determine the action potential propagating speed from a different IMD in order to coordinate the delivery of other simulation signals as described herein.

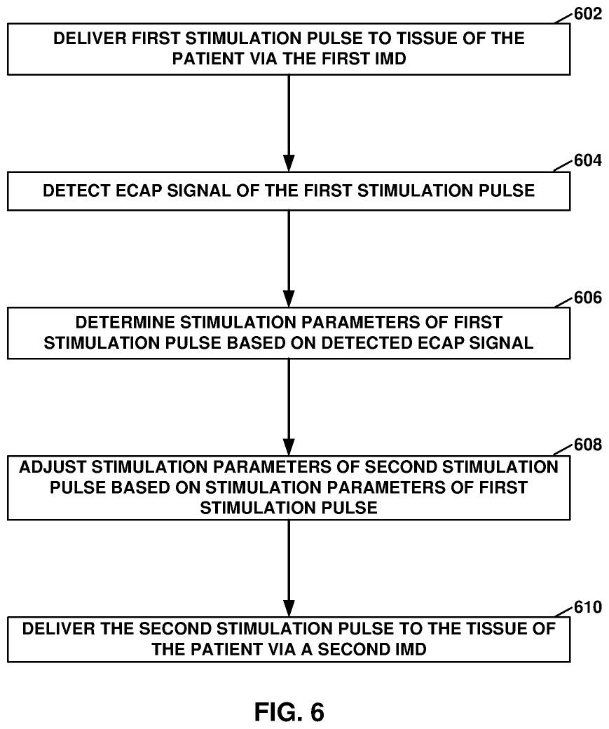

FIG. 6 is a flow diagram illustrating an example process for controlling simulation based on a plurality of stimulation signals by system 100 including a plurality of IMDs (e.g., IMDs 106 of FIG. 1), in accordance with one or more techniques of this disclosure. While FIG. 6 describes system 100 with reference to a first IMD (e.g., IMD 106A of FIG. 1) and a second IMD (e.g., IMD 106B of FIG. 1), system 100 may include three or more IMDs, two or more IMDs and an external programmer (e.g., external programmer 120), or the like.

System 100 may deliver a first stimulation pulse to tissue of patient 102 via first IMD 106A (602). First IMD 106A may deliver the first stimulation pulse to a first target tissue of patient 102, e.g., to target nerves within the target tissue. The first stimulation pulse may deliver electrical stimulation and/or medical therapy to the target tissue of patient 102. The first stimulation pulse may include a control pulse and/or a base pulse (e.g., base pulse 404, base pulse 412) as previously described herein. In some examples, the first stimulation pulse may have an intensity greater than an action potential threshold and/or a perception threshold of target tissue of patient 102. First IMD 106A may continue to deliver the first stimulation pulse to the tissue in accordance with the stimulation parameters of the first stimulation pulse until first IMD 106A receives an instruction to terminate stimulation, until a predetermined period of time has elapsed, or until a predetermined number of first stimulation pulses have been delivered.

First IMD 106A may deliver the first stimulation pulse to the tissue of patient 102 via electrodes (e.g., electrodes 220, 222) disposed on leads 108. Two or more electrodes may define a bipolar connection forming a cathode-anode pair. First IMD 106A may transmit electrical signals defining the first stimulation pulse from a cathode of the electrode pair, through the tissue of patient 102, and into an anode of the electrode pair. In some examples, the electrodes of first IMD 106A may define a multipolar electrode connection and deliver the first stimulation pulse to the tissue via two or more electrodes of the multipolar electrode connection.

In some examples, first IMD 106A may automatically deliver the first stimulation pulse to the tissue of patient 102. First IMD 106A may detect a signal and/or stimulus in the target tissue patient 102 via electrodes on leads 108 coupled to first IMD 106A, sensor(s) of first IMD 106A (e.g., sensor(s) 222), or the like. Processing circuitry of first IMD 106A (e.g., processing circuitry 210) and/or sensing circuitry of first IMD 106A (e.g., sensing circuitry 206) may determine that the detected signal and/or stimulus satisfies a threshold condition corresponding to an occurrence of a medical condition and deliver the first stimulation pulse in response to the determination.

In some examples, first IMD 106A may select a therapy stimulation program from a plurality of therapy stimulation programs (e.g., therapy stimulation programs 214) stored in a storage device (e.g., storage device 212) of first IMD 106A. For example, first IMD 106A may determine a type and/or severity of a medical condition experienced by patient 102 and select a therapy stimulation program configured to treat the medical condition based on the determined type and severity. For example, first IMD 106A may determine a level of pain experienced by patient 102 and select a therapy stimulation program based on the level. Each therapy stimulation program may include stimulation parameters for the first stimulation pulse. First IMD 106A may apply the selected therapy stimulation program and deliver the first stimulation pulse in accordance with the selected therapy stimulation program.

In some examples, external programmer 120 may receive user input via a user interface (e.g., user interface 306). The user input may include, but is not limited to, a type of medical condition experienced by patient 102, a type of medical therapy desired by patient 102, a desired effect of a delivered medical therapy, an undesired side effect (e.g., paresthesia) of delivered medical therapy, a localization of a side effect of the delivered medical therapy or the like. For example, patient 102 may input, via user interface 306, a indication to not experience a particular side effect or to localize the particular side effect to a particular portion of the body of patient 102 (e.g., to a limb). Based on the user input, external programmer 120 and/or first IMD 106A may selected an appropriate therapy stimulation program and first IMD 106A may deliver the first stimulation pulse in accordance with the selected therapy stimulation program.

System 100 may detect ECAP signals of the first stimulation pulse (604). In some examples the first stimulation pulse may exceed a detection threshold or an action potential threshold in nerves within the target tissue of patient 102. Delivery of the first stimulation pulse to the tissue by first IMD 106A causes the nerves in the tissue to generate a resulting action potential (e.g., an ECAP signal). The ECAP signal may travel along the nerve and may be detected by one or more IMDs. The one or more IMDs may include a second IMD 106B configured to deliver electrical stimulation to patient 102, e.g., to another target tissue of patient 102. Second IMD 106B may sense the ECAP signals via electrodes (e.g., electrodes 220, 222) disposed on one or more leads 108 connected to second IMD 106B. In some examples, second IMD 106B may sense the ECAP signals via one or more sensor(s) 222 coupled to second IMD 106B.

In some examples, the one or more IMDS include a third IMD configured to sense the ECAP signals and transmit the sensed ECAP signals to first IMD 106A and second IMD 106B. In some examples, the third IMD may transmit the sensed ECAP signals to external programmer 120.

In some examples, the first stimulation pulse may be a subthreshold signal (i.e., an electrical signal below the detection threshold and/or action potential threshold). In such examples, system 100 may detect, via one or more IMDs, a stimulation signal in the nerve(s) of patient 102. The stimulation signal may correspond to the first stimulation pulse.

System 100 may determine stimulation parameters of the first stimulation pulse based on the detected ECAP signal (606). In some examples, second IMD 106B may determine the stimulations parameters of the first stimulation pulse. In some examples, external programmer 120 may receive the sensed ECAP signals from second IMD 106B and determine the stimulation parameters of the first stimulation pulse. In some examples, another IMD in communication with first IMD 106A and second IMD 106B may receive the sensed ECAP signals (e.g., from second IMD 106B, from a third IMD) and determine the stimulation parameter of the first stimulation pulse.

System 100 may determine, based on nerve conduction velocities (e.g., measured or estimated nerve conduction velocities), and the timing of the detected ECAP signals, the timing of the first stimulation pulse. In some examples, system 100 may determine, based on the width of peaks in the detected ECAP signal and/or a number of peaks in the detected ECAP signal, a pulse width of the first stimulation pulse. In some examples, system 100 may determine, based on a frequency of the detected ECAP signals a frequency (e.g., and thereby the timing) of the first stimulation pulse.

System 100 may determine a signal width, a signal amplitude, or a signal frequency of the ECAP signal and may determine the stimulation parameters of the first stimulation pulse, e.g., as described above, based on one or more of the determined signal width, signal amplitude, or signal frequency of the ECAP signal. In some examples, System 100 may receive a distance between first IMD 106A and second IMD 106B and a nerve conduction velocity of patient 102. For example, system 100 may determine, based on the detected ECAP signal and the distance between the electrodes of first IMD 106A and second IMD 106B, an average nerve conduction velocity at which tissue at a specific location on patient 102 may conduct action potential signals. Based on the received distance, the nerve conduction velocity, and the sensed ECAP signal, system 100 may determine one or more timing parameters of the first stimulation pulse. The timing parameters include, but are not limited to, a start-of-delivery time for the first stimulation pulse, an end-of-delivery time for the first stimulation pulse, a duty cycle of the first stimulation pulse, a pulse frequency of the first stimulation pulse, a length of any of a cycle (e.g., cycle 410), a segment of the cycle (e.g., segment 407), a length of a slot (e.g., slot 408) of one of the segments, a time period between adjacent slots, or the like.

In some examples, system 100 may determine a distance between the electrodes of first IMD 106A and the electrodes of second IMD 106B. The distance may be a linear distance or a distance along nerves connecting a first target region corresponding to first IMD 106A and a second target region corresponding to second IMD 106B. System 100 may also determine a first time when first IMD 106A delivers the first stimulation pulse and a second time when second IMD 106B detects the ECAP signal. Based on the determined distance, the first time, and the second time, system 100 may determine a nerve conduction velocity of patient 102.

System 100 may adjust stimulation parameters of a second stimulation pulse based on stimulation parameters of the first stimulation pulse (608). System 100 may determine, based on one or more of the simulation parameters of a selected therapy stimulation program or user input, a target timing of the first stimulation pulse and the second stimulation pulse relative to each other. For example, system 100 may cause first IMD 106A and second IMD 106B to deliver the stimulation pulses simultaneously, to stagger the delivery of the stimulation pulses, to amplify, propagate, diminish, or cancel out one stimulation pulse via delivery of the other stimulation pulse, or the like.

System 100 may, based on the determined timing parameters of the first stimulation pulse, adjust the stimulation parameters (e.g., the timing parameters) of the second stimulation pulse to comply with the target timing of the first stimulation pulse and the second stimulation pulse. For example, based on the timing parameters of the first stimulation pulse, system 100 may determine a time to cause second IMD 106B to deliver the second stimulation pulse such that a time difference between the first stimulation pulse and the second stimulation pulse satisfies the target timing. For example, if the target timing includes simultaneous delivery of the first and second stimulation pulses, system 100 (e.g., second IMD 106B, external programmer 120) may adjust the timing parameter so the second stimulation pulse. The adjusted timing parameters of the second stimulation pulse may cause the second IMD 106B to deliver the second stimulation pulse at a same time as when first IMD 106A delivers the first stimulation pulse. System 100 may determine, based on the timing parameters of the first stimulation pulse, a third time when first IMD 106A will deliver the next first stimulation pulse. System 100 may then adjust the timing parameters of the second stimulation pulse to cause second IMD 106B to deliver the second stimulation pulse at the third time.

In some examples, the target timing may include a time of arrival of the first stimulation pulse and the second stimulation pulse at a target region of patient 102. In such examples, system 100 adjusts the timing parameters of the second stimulation pulse to account for the nerve conduction velocity of patient 102. For example, second IMD 106B may be instructed to deliver the second stimulation pulse at an earlier or later time than when first IMD 106A delivers the first stimulation pulse. Due to the distances between IMDs 106 and the target region and the nerve conduction velocity of patient 102, the first and second stimulation pulses would arrive at the target region at the same time.

System 100 may deliver the second stimulation pulse to the tissue of patient 102 via second IMD 106B (610). Second IMD 106B may deliver the second stimulation pulse to the tissue via electrodes disposed on leads 108 connected to second IMD 106B. In some examples, second IMD 106B may deliver the second stimulation pulse via electrodes disposed on the housing of second IMD 106B or otherwise connected to circuitry of second IMD 106B. Second IMD 106B may deliver the second stimulation pulse to the tissue until second IMD 106B receives an instruction (e.g., from external programmer 120, from IMD 106A) to terminate stimulation, until a predetermined period of time has elapsed, or until a predetermined number of second stimulation pulses have been delivered.

Over time, the timing parameters of the stimulation pulses may change and/or the nerve conduction velocity of patient 102 may change. The changes may be due to changes in physiology of patient 102, a progression in disease state of patient 102, changes in the selected therapy stimulation program, or the like. System 100 (e.g., second IMD 106B, external programmer 120, another IMD) may continue to monitor ECAP signals resulting from the delivered first stimulation pulses. If system 100 detects deviations from the target timing of the first stimulation pulse and the second stimulation pulse, system 100 may adjust stimulation parameters of one or more of the first stimulation pulse or the second stimulation pulse, e.g., to achieve the target timing of the stimulation pulses.

In some times, second IMD 106B, external programmer 120 and/or another IMD may control first IMD 106A. In such examples, one or more of second IMD 106B, external programmer 120, and/or another IMD may adjust stimulation parameters of the first stimulation pulse, e.g., to achieve the target timing, to adjust the target timing. The devices may then transmit instructions to first IMD 106A to cause first IMD 106A to deliver the first stimulation pulses based on the adjusted stimulation parameters of the first stimulation pulse.

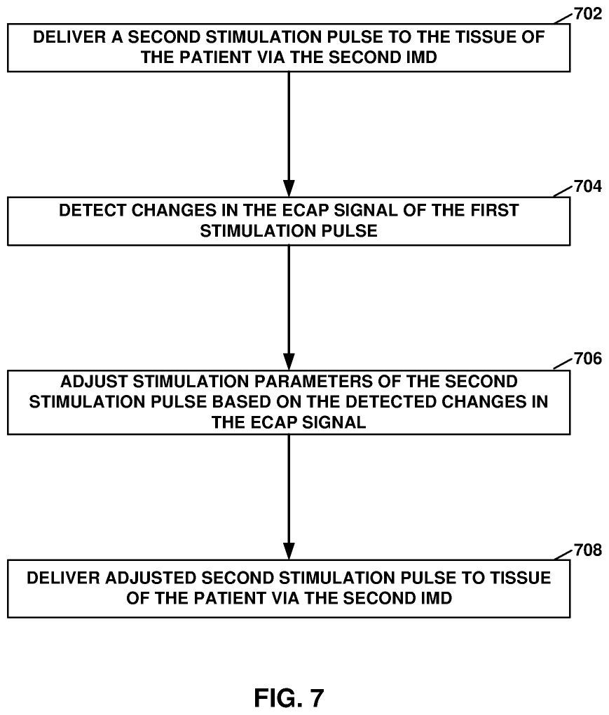

FIG. 7 is a flow diagram illustrating an example process for adjusting a simulation pulse in response to changes in the ECAP signal, in accordance with one or more techniques of this disclosure. While FIG. 7 is described primarily with reference to system 100 adjusting a second stimulation pulse based on changes to an ECAP signal of a first stimulation pulse, the example process of FIG. 7 may be applied to adjust the first stimulation pulse based on changes to an ECAP signal of the second stimulation pulse.

System 100 may deliver a second stimulation pulse to the tissue of patient 102 via second IMD 106B (702). During delivery of and/or after the delivery of the second stimulation pulse, System 100 (e.g., second IMD 106B, external programmer 120, and/or another IMD of system 100) may detect changes in the ECAP signal of the first stimulation pulse (704). The changes may include changes in the parameters of the ECAP signal such as, but are not limited to, changes to a signal width, a signal amplitude, or a signal frequency of the detected ECAP signal. The changes in the ECAP signal may be a result of changes in the physiology of patient 102 and/or in the disease state of patient 102. In some examples, sensing circuitry of system 100 (e.g., sensing circuitry 206 of IMD 200) may detect the ECAP signals via electrodes (e.g., electrodes 220, 222) disposed on leads (e.g., leads 108) connected to the sensing circuitry. The sensing circuitry may then compare the detected ECAP signals with prior detected ECAP signals stored in a storage device of system 100 (e.g., storage device 212, storage device 304) to determine the changes in the ECAP signal of the first stimulation pulse.

System 100 may adjust stimulation parameters of the second stimulation pulse based on the detected changes in the ECAP signal (706). In some examples, system 100 may determine, based on the changes in the ECAP signal, a change in the nerve conduction velocity of patient 102 (e.g., due to changes in the nerves connecting the first target tissue to the second target tissue). System 100 may then adjust stimulation parameters of the second stimulation pulse based on the determined change in the nerve conduction velocity. System 100 may change the timing parameters of the second stimulation pulses such that a target tissue (e.g., the second target tissue) may receive the first stimulation pulse and the second stimulation pulse within a predetermined time period of each other in accordance with the selected therapy stimulation program. For example, system 100 can reduce or increase a time period between delivering the first stimulation pulse by first IMD 106A and delivering the second stimulation pulse by second IMD 106B to account for the change in the nerve conduction velocity. The second target tissue may then receive the first stimulation pulse and the second stimulation pulse simultaneously, e.g., in accordance with a selected therapy stimulation program. In some examples, system 100 may coordinate delivery of the first stimulation pulse and/or the second stimulation pulse with an action potential signal of one or more of the first stimulation pulse or the second stimulation pulse, e.g., to propagate or interfere with the transmission of the action potential signal along a nerve of patient 102.

In some examples, system 100 may determine, based on the changes in the ECAP signal, that patient 102 requires and/or desires a different therapy stimulation program. System 100 may then select a new therapy stimulation program and adjust the stimulation parameters of the second stimulation pulse based on the new therapy stimulation program. System 100 may adjust the timing parameters of the second stimulation pulse such that the timing between the first stimulation pulse and the second stimulation pulse satisfies a predetermined timing of the new therapy stimulation program. For example, system 100 may adjust the timing parameters of the second stimulation pulse to cause second IMD 106B to deliver the second stimulation pulse to a target tissue within a predetermined time period of the delivery of the first stimulation pulse and/or receipt of an ECAP signal of the first stimulation pulse by the target tissue, in accordance with the predetermined timing of the new therapy stimulation program.

System 100 may then deliver the adjusted second stimulation pulse to tissue of patient 102 via second IMD 106B (708). Second IMD 106B may generate the electrical signals of the second stimulation pulse via stimulation generation circuitry (e.g., stimulation generation circuitry 202) and deliver the electricals signals to the target tissue via electrodes on lead 108C at a predetermined time. The predetermined time may be based on the adjusted timing parameters for the second stimulation pulse.

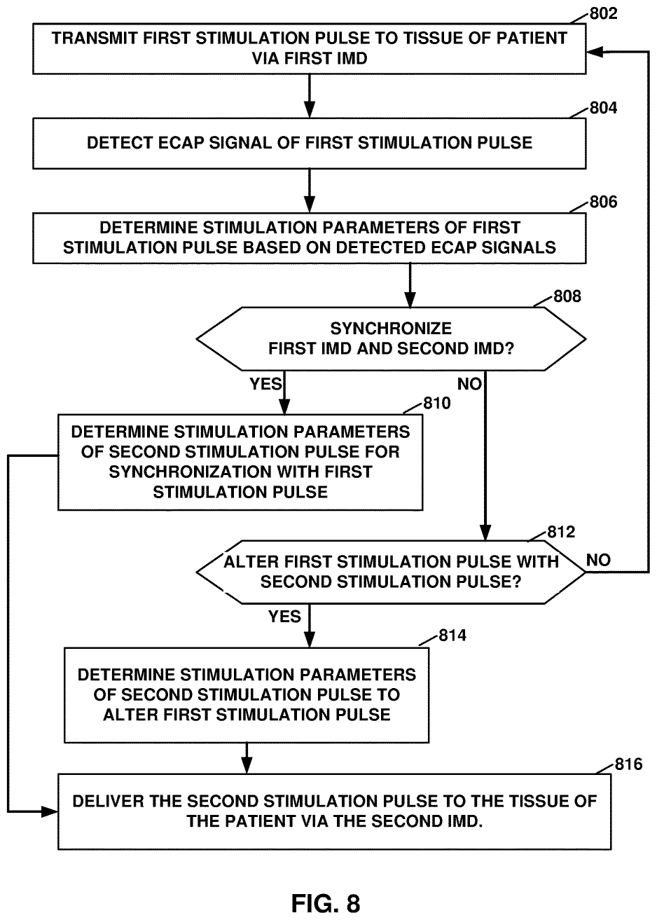

FIG. 8 is a flow diagram illustrating another example process for controlling stimulation based on a plurality of stimulation signals by a plurality of IMDs (e.g., first IMD 106A, second IMD 106B), in accordance with one or more techniques of this disclosure. While FIG. 8 describes system 100 with reference to a first IMD (e.g., IMD 106A of FIG. 1) and a second IMD (e.g., IMD 106B of FIG. 1), system 100 may include three or more IMDs, two or more IMDs and an external programmer (e.g., external programmer 120), or the like.

System 100 may transmit a first stimulation pulse to tissue of patient 102 via first IMD 106A (802). First IMD 106A may transmit the first stimulation pulse to the tissue via electrodes 220, 222 disposed on leads 108A, 108B, e.g., as previously described herein. System 100 may detect an ECAP signal of the first stimulation pulse (804) and may determine stimulation parameters of the first stimulation pulse based on the detected ECAP signals (806). For example, system 100 may determining the timing of delivery, pulse width, and/or pulse frequency of the first stimulation pulse based on the detected ECAP signals. One or more components of system 100 including external programmer 120, second IMD 106B, and/or another IMD may perform steps 804 and 806 in accordance with example methods and processes previously described herein.

System 100 may determine whether to synchronize first IMD 106A and second IMD 106B (808). Synchronizing first IMD 106A and second IMD 106B may cause second IMD 106B to deliver the second stimulation pulse at the same time as first IMD 106A, for the first stimulation pulse and the second stimulation pulse to arrive at a target tissue (e.g., at a specific nerve) at the same time, or for the second stimulation pulse and the ECAP signal of the first stimulation pulse to arrive at the target tissue at the same time. In some examples, simultaneous delivery or reception of the stimulation pulses may amplify one of the stimulation pulses (e.g., the first stimulation pulses) and/or may extend the area of influence of the first stimulation pulse. In some examples, the second stimulation pulse may propagate or impede transmission of the ECAP signal of the first stimulation pulse through the target tissue.

System 100 may determine whether to synchronize IMDs 106 based on user input received by external programmer 120 via user interface 306. In some examples system 100 may make the determination based on prior user input received by system 100. In some examples, system 100 may apply a first machine learning model to determine whether to synchronize IMDs 106. The first machine learning model may be trained with a training set including past stimulation parameters of the first stimulation pulse, past sensor data for medical conditions, and prior selection by patients (e.g., patient 102, other patients with similar physiology). The training set may indicate prior selection by patients based on the medical condition experienced by the patient at the time (e.g., a type of pain experienced by the patient) and the stimulation parameters of the first stimulation pulse delivered by system 100. System 100 may apply the first machine learning model by inputting stimulation parameters of the first stimulation pulse and sensor data from sensors on IMDs 106 to output a determination of whether to synchronize IMDs 106.

Based on a determination to synchronize first IMD 106A and second IMD 106B (“YES” branch of step 808), system 100 may determine stimulation parameters of second stimulation pulse for synchronization with first stimulation pulse (810). System 100 may select a therapy stimulation program from a plurality of therapy stimulation programs 214 with a predetermined timing synchronizing both IMDs 106 and with other stimulation parameters corresponding to stimulation therapy required and/or desired by patient 102. System 100 (e.g., external programmer 120, second IMD 106B) may then adjust stimulation parameters of the second stimulation pulse in accordance with the selected therapy stimulation program. For example, system 100 may adjust the timing parameters of a default second stimulation pulse stored in second IMD 106B such that when second IMD 106B is delivering the second stimulation pulse in accordance with the stimulation parameters, second IMD 106B delivers the second stimulation pulse at a same time as when first IMD 106A delivers the first stimulation pulse.

System 100 may then deliver the second stimulation pulse to the tissue of patient 102 via second IMD 106B (816). Second IMD 106B may deliver the second stimulation pulse based on the determined stimulation parameters and in accordance with the example methods and/or processes previously described herein.

Based on a determination not to synchronize first IMD 106A and second IMD 106B (“NO” branch of step 808), system 100 may determine whether to alter the first stimulation pulse with the second stimulation pulse (812). In some examples, second IMD 106B may deliver a second stimulation pulse between two adjacent first stimulation pulses, e.g., to reduce power consumption by either of IMDs 106. In some examples, second IMD 106B may deliver the second stimulation pulse at an amount of time after delivery of the first stimulation pulse by the first IMD 106A, e.g., to amplify, to propagate, to diminish, or to cancel out the first stimulation pulse. For example, the second stimulation pulse may constructively interfere with the first stimulation pulse to propagate or amplify the first stimulation pulse. In another example, the second stimulation pulse may destructively interfere with the first stimulation pulses to diminish or cancel out the first stimulation pulse. In some examples, the second stimulation pulse may constructively or destructively interfere with an action potential signal (e.g., an ECAP signal) of the first stimulation pulse to propagate, diminish, or cancel out the action potential signal.

System 100 may determine whether to alter the first stimulation pulse based on user input. For example, patient 102 may select a therapy stimulation program that alters the first stimulation pulse. In some examples, patient 102 may select a desired therapy result (e.g., limited paresthesia, no paresthesia) and system 100 determine whether to alter the first stimulation pulse based on the desired therapy result. For example, if patient 102 indicates limited therapy, such as paresthesia, to a specific area (e.g., a limb) or undesired side effect via a user interface (e.g., user interface 306) of programmer 120, system 100 may determine to alter the first stimulation pulse by causing the second stimulation pulse to cancel out the first stimulation pulse outside of the specified area.

In some examples, system 100 may apply a second machine learning model to determine whether to alter the first stimulation pulse. The second machine learning model may be trained with a training set including past stimulation parameters of the first stimulation pulse, past sensor data for medical conditions, and prior selection by patients (e.g., patient 102, other patients with similar physiology). The training set may indicate prior selection by patients to alter the first stimulation pulse and/or how to alter the first stimulation pulse based on the medical condition experienced by the patient at the time (e.g., a type of pain experienced by the patient) and the stimulation parameters of the first stimulation pulse delivered by system 100. System 100 may apply the first machine learning model by inputting stimulation parameters of the first stimulation pulse and sensor data from sensors on IMDs 106 to output a determination of whether to alter the first stimulation pulse. In some examples, the second machine learning model may also output a type of alteration (e.g., propagation, diminishment, amplification, cancellation) and/or an amount of alteration of the first stimulation pulse by the second stimulation pulse. The amount of alteration of the first stimulation pulse may be measured by the change in the pulse amplitude of the first stimulation pulse within a target tissue of patient 102.

Based on a determination not to alter the first stimulation pulse with the second stimulation pulse (“NO” branch of 812), system 100 may continue to transmit the first stimulation pulse to the tissue of patient 102 via first IMD 106A (802). Based on a determination to alter the first stimulation pulse and/or an action potential signal of the first stimulation with the second stimulation pulse (“YES” branch of 812), system 100 may determine stimulation parameters of second stimulation pulse to alter the first stimulation pulse (814). In some examples, system 100 (e.g., external programmer 120, second IMD 106B, and/or another IMD) determines the stimulation parameters based at least in part on the type of alteration and/or an amount of alteration of the first stimulation pulse. For example, based on a determination to diminish the first stimulation pulse by a greater amount, system 100 may determine the timing parameters of the second stimulation pulse to destructively interfere with the first stimulation pulse and increase the pulse amplitude of the second stimulation pulse. System 100 may then deliver the second stimulation pulse to the tissue of the patient via second IMD 106B (816). In some examples, system 100 may perform the process illustrated in FIG. 8 to propagate or diminish an action potential pulse along a nerve of patient 102.

The following examples are example systems, devices, and methods described herein.

Example 1: a system comprising: a first implantable medical device (IMD) implanted within a first tissue of a patient, the first IMD comprising; sensing circuitry configured to generate sensed information representative of an evoked electrical signal elicited by a first stimulation signal delivered from a second IMD different from the first IMD to a second tissue of the patient, the second tissue being at a different location than the first tissue; and processing circuitry configured to: determine, based on the sensed information representative of the evoked electrical signal, one or more stimulation parameters at least partially defining a second stimulation signal; and control stimulation generation circuitry of the first IMD to deliver the second stimulation signal to the first tissue of the patient according to the one or more stimulation parameters.

Example 2: the system of example 1, wherein the sensed information representative of the evoked electrical signal comprises one or more of a signal width, a signal amplitude, or a signal frequency of the evoked electrical signal.

Example 3: the system of any of examples 1 and 2, wherein the second stimulation signal increases the propagation of the first stimulation signal within the first tissue.

Example 4: the system of any of examples 1 and 2, wherein the second stimulation signal at least partially diminishes the propagation of the first stimulation signal within the first tissue.

Example 5: the system of example 4, wherein the second stimulation signal at least partially cancels out the propagation of the first stimulation signal within the first tissue of the patient.

Example 6: the system of any of examples 1-5, wherein the first IMD comprises a peripheral nerve stimulation (PNS) device.

Example 7: the system of any of examples 1-6, wherein the first IMD comprises a spinal cord stimulation (SCS) device.

Example 8: the system of any of examples 1-7, wherein the first IMD comprises a PNS device and the second IMD comprises a SCS device.

Example 9: the system of any of examples 1-8, wherein the one or more stimulation parameters of the second stimulation signal comprises one or more of a signal width, a signal amplitude, or a signal frequency of the second stimulation signal.

Example 10: the system of any of examples 1-9, wherein the processing circuitry is further configured to: determine one or more time periods during which the second IMD does not deliver the first stimulation signal to the second tissue; determine the one or more stimulation parameters of the second stimulation signal corresponding to transmission of the second stimulation signal to the first tissue during the one or more time periods; and control the stimulation generation circuitry to deliver the second stimulation signal to the first tissue during the one or more time periods.

Example 11: the system of any of examples 1-10, wherein the processing circuitry is further configured to: determine a change in the sensed information representative of the evoked electrical signal; and adjust the one or more stimulation parameters of the second stimulation signal based on the determined change in the sensed information.

Example 12: the system of any of examples 1-11, wherein the first IMD further comprises communications circuitry configured to establish communications between the first IMD and the second IMD, and wherein the processing circuitry is further configured to: determine, via the communications circuitry, a first time when the second IMD transmits the first stimulation signal; determine, via the sensing circuitry, a second time when the first IMD sensed the evoked electrical signal; determine, based on the first time and the second time, a nerve conduction velocity of the patient; and determine the one or more stimulation parameters based at least in part on the determined nerve conduction velocity.

Example 13: the system of any of examples 1-12, wherein the evoked electrical signal comprises an evoked compound action potential (ECAP) signal.

Example 14: the system of any of examples 1-13, further comprising an external device in communication with the first IMD, and wherein the processing circuitry is further configured to: receive, via the external device, an indication from the patient of the effect of the transmitted second stimulation signal on a symptom; adjust the one or more stimulation parameters of the second stimulation signal based on the received indication; and control the signal generation circuitry to deliver the second stimulation signal to the first tissue according to the adjusted one or more stimulation parameters.

Example 15: a method comprising: sensing, via sensing circuitry of a first implantable medical device (IMD) implanted within a first tissue of a first region of a patient, information representative of an evoked electrical signal elicited by a first stimulation signal delivered from a second IMD different from the first IMD to a second tissue of a second region of the patient; determining, by processing circuitry of the first IMD and based on the sensed information representative of the evoked electrical signal, one or more stimulation parameters at least partially defining a second stimulation signal; and controlling, by the processing circuitry, stimulation generation circuitry of the first IMD to deliver the second stimulation signal to the first tissue according to the one or more stimulation parameters.

Example 16: the method of example 15, wherein the sensed information representative of the evoked electrical signal comprises one or more of a signal width, a signal amplitude, or a signal frequency of the evoked electrical signal.

Example 17: the method of any of examples 15 and 16, wherein the second stimulation signal improves the propagation of the first stimulation signal within the first tissue.

Example 18: the method of any of examples 15 and 16, wherein the second stimulation signal at least partially diminishes the propagation of the first stimulation signal within the first tissue.

Example 19: the method of example 18, wherein the second stimulation signal at least partially cancels out the propagation of the first stimulation signal within the first tissue.

Example 20: the method of any of examples 15-19, wherein the first IMD comprises a peripheral nerve stimulation (PNS) device.

Example 21: the method of any of examples 15-20, wherein the first IMD comprises a spinal cord stimulation (SCS) device.

Example 22: the method of any of examples 15-21, wherein the first IMD comprises a PNS device and the second IMD comprises a SCS device.

Example 23: the method of any of examples 15-22, wherein the one or more stimulation parameters of the second stimulation signal comprises one or more of a signal width, a signal amplitude, or a signal frequency of the second stimulation signal.

Example 24: the method of any of examples 15-23, further comprising: determining, by the processing circuitry, one or more time periods during which the second IMD does not deliver the first stimulation signal to the second tissue; determining, by the processing circuitry, the one or more stimulation parameters of the second stimulation signal corresponding to transmission of the second stimulation signal to the first tissue during the one or more time periods; and controlling the stimulation generation circuitry to deliver the second stimulation signal to the first tissue during the one or more time periods.

Example 25: the method of any of examples 15-24, further comprising: determining, by the processing circuitry, a change in the sensed information representative of the evoked electrical signal; and adjusting, by the processing circuitry, the one or more stimulation parameters of the second stimulation signal based on the determined change in the sensed information.

Example 26: the method of any of examples 15-25, further comprising: determining, by communications circuitry of the first IMD, a first time when the second IMD transmits the first stimulation signal; determining, by the sensing circuitry, a second time when the first IMD detects the evoked electrical signal; determining, by the processing circuitry and based on the first time and the second time, a nerve conduction velocity of the patient; and determining, by the processing circuitry, the one or more stimulation parameters based at least in part on the determined nerve conduction velocity.

Example 27: the method of any of examples 15-26, wherein the evoked electrical signal comprises an evoked compound action potential (ECAP) signal.

Example 28: the method of any of examples 15-27, further comprising:
receiving, by the processing circuitry and from an external device in communication with the first IMD, an indication from the patient of the effect of the delivered second stimulation signal on a symptom; adjusting, by the processing circuitry, the one or more stimulation parameters of the second stimulation signal based on the received indication; and controlling, by the processing circuitry, the stimulation generation circuitry to deliver the second stimulation signal to the first tissue according to the adjusted one or more stimulation parameters.

Example 29: a computer-readable medium comprising instructions that, when executed, cause processing circuitry of an implantable medical device (IMD) to perform the method of any of examples 15-28.

Example 30: a system comprising: an external device comprising: communications circuitry configured to be in communications with a first implanted medical device (IMD) implanted within a first tissue of a patient and a second IMD implanted within a second tissue of the patient, the second tissue being at a different location than the first tissue; and processing circuitry configured to: receive, via the communications circuitry and from the second IMD, stimulation information representative of a first stimulation signal delivered from the second IMD to the second tissue; receive, via the communications circuitry and from the first IMD, sensed information representative of an evoked electrical signal elicited by the first stimulation signal; determine, based on the received stimulation information and the received sensed information, one or more stimulation parameters at least partially defining a second stimulation signal; and transmit, via the communications circuitry, instructions to the first IMD to deliver the second stimulation signal to the first tissue according to the one or more stimulation parameters.

Example 31: the system of example 30, wherein the sensed information representative of the evoked electrical signal comprises one or more of a signal width, a signal amplitude, or a signal frequency of the evoked electrical signal.

Example 32: the system of any of examples 30 and 31, wherein the second stimulation signal increases the propagation of the first stimulation signal within the first tissue.

Example 33: the system of any of examples 30 and 31, wherein the second stimulation signal at least partially diminishes the propagation of the first stimulation signal within the first tissue.

Example 34: the system of example 28, wherein the second stimulation signal at least partially cancels out the propagation of the first stimulation signal within the first tissue.

Example 35: the system of any of examples 30-34, wherein at least one of the first IMD or the second IMD comprises a peripheral nerve stimulation (PNS) device.

Example 36: the system of any of examples 30-35, wherein at least one of the first IMD or the second IMD comprises a spinal cord stimulation (SCS) device.

Example 37: the system of any of examples 30-36, wherein the first IMD comprises a PNS device and the second IMD comprises a SCS device.

Example 38: the system of any of examples 30-37, wherein the one or more stimulation parameters comprises one or more of a signal width, a signal amplitude, or a signal frequency of the second stimulation signal.

Example 39: the system of any of examples 30-38, wherein the stimulation information comprises one or more of a signal width, a signal amplitude, a signal frequency, or a signal delivery time of the first stimulation signal.

Example 40: the system of any of examples 30-39, wherein the processing circuitry is configured to: determine, based on the received stimulation information, one or more time periods during which the second IMD does not deliver the first stimulation signal to the second tissue; determine, based on the received sensed information representative of the evoked electrical signal, the one or more stimulation parameters of the second stimulation signal corresponding to transmission of the second stimulation signal to the first tissue during the one or more time periods; and transmit, via the communications circuitry, instructions to the first IMD to deliver the second stimulation signal to the first tissue during the one or more time periods.

Example 41: the system of any of examples 30-40, wherein the processing circuitry is configured to: determine a change in the sensed information representative of the evoked electrical signal; and adjust the one or more stimulation parameters of the second stimulation signal based on the determined change in the sensed information.

Example 42: the system of any of examples 30-41, wherein the processing circuitry is configured to: determine, based on the received stimulation information, a first time when the second IMD transmits the first stimulation signal; determine, based on the received sensed information, a second time when the first IMD senses the evoked electrical signal; determine, based on the first time and the second time, a nerve conduction velocity of the patient; and determine the one or more stimulation parameters based at least in part on the determined nerve conduction velocity.

Example 43: the system of any of examples 30-42, wherein the evoked electrical signal comprises an evoked compound action potential (ECAP) signal.

Example 44: the system of any of examples 30-43, wherein the external device further comprises a user interface (UI), and wherein the processing circuitry is configured to: receive, via the UI, an indication from the patient of the effect of the delivered second stimulation signal on a symptom; adjust the one or more stimulation parameters of the second stimulation signal based on the received indication; and transmit, via the communications circuitry, instructions to the first IMD to deliver the second stimulation signal to the first tissue according to the adjusted one or more stimulation parameters.

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

1-15. (canceled)

16. A system comprising:
a first implantable medical device (IMD) implanted within a first tissue of a patient, the first IMD comprising;
sensing circuitry configured to generate sensed information representative of an evoked electrical signal elicited by a first stimulation signal delivered from a second IMD different from the first IMD to a second tissue of the patient, the second tissue being at a different location than the first tissue; and
processing circuitry configured to:
determine, based on the sensed information representative of the evoked electrical signal, one or more stimulation parameters at least partially defining a second stimulation signal; and
control stimulation generation circuitry of the first IMD to deliver the second stimulation signal to the first tissue of the patient according to the one or more stimulation parameters.

17. The system of claim 16, wherein the sensed information representative of the evoked electrical signal comprises one or more of a signal peak width, a signal amplitude, or a signal frequency of the evoked electrical signal.

18. The system of claim 16, wherein the second stimulation signal increases propagation of the first stimulation signal or the evoked electrical signal within the first tissue.

19. The system of claim 16, wherein the second stimulation signal at least partially diminishes propagation of the first stimulation pulse or the evoked electrical signal within the first tissue.

20. The system of claim 19, wherein the second stimulation signal at least partially cancels out propagation of the first stimulation signal or the evoked electrical signal within the first tissue of the patient.

21. The system of claim 16, wherein the one or more stimulation parameters of the second stimulation signal comprises one or more of a signal width, a signal amplitude, or a signal frequency of the second stimulation signal.

22. The system of claim 16, wherein the processing circuitry is further configured to:
determine one or more time periods during which the second IMD does not deliver the first stimulation signal to the second tissue;
determine the one or more stimulation parameters of the second stimulation signal corresponding to transmission of the second stimulation signal to the first tissue during the one or more time periods; and
control the stimulation generation circuitry to deliver the second stimulation signal to the first tissue during the one or more time periods.

23. The system of claim 16, wherein the processing circuitry is further configured to:
determine a change in the sensed information representative of the evoked electrical signal; and
adjust the one or more stimulation parameters of the second stimulation signal based on the determined change in the sensed information.

24. The system of claim 16, wherein the first IMD further comprises communications circuitry configured to establish communications between the first IMD and the second IMD, and wherein the processing circuitry is further configured to:
determine, via the communications circuitry, a first time when the second IMD transmits the first stimulation signal;
determine, via the sensing circuitry, a second time when the first IMD sensed the evoked electrical signal;
determine, based on the first time and the second time, a nerve conduction velocity of the patient; and
determine the one or more stimulation parameters based at least in part on the determined nerve conduction velocity.

25. The system of claim 16, wherein the evoked electrical signal comprises an evoked compound action potential (ECAP) signal.

26. The system of claim 16, further comprising an external device in communication with the first IMD, and wherein the processing circuitry is further configured to:
receive, via the external device, an indication from the patient of the effect of the transmitted second stimulation signal on a symptom;
adjust the one or more stimulation parameters of the second stimulation signal based on the received indication; and
control the stimulation generation circuitry to deliver the second stimulation signal to the first tissue according to the adjusted one or more stimulation parameters.

27. A method comprising:
sensing, via sensing circuitry of a first implantable medical device (IMD) implanted within a first tissue of a first region of a patient, information representative of an evoked electrical signal elicited by a first stimulation signal delivered from a second IMD different from the first IMD to a second tissue of a second region of the patient;
determining, by processing circuitry of the first IMD and based on the sensed information representative of the evoked electrical signal, one or more stimulation parameters at least partially defining a second stimulation signal; and
controlling, by the processing circuitry, stimulation generation circuitry of the first IMD to deliver the second stimulation signal to the first tissue according to the one or more stimulation parameters.

28. The method of claim 27, wherein the sensed information representative of the evoked electrical signal comprises one or more of a signal peak width, a signal amplitude, or a signal frequency of the evoked electrical signal.

29. The method of claim 27, wherein the second stimulation signal improves the propagation of the first stimulation signal or the evoked electrical signal within the first tissue.

30. The method of claim 27, wherein the second stimulation signal at least partially diminishes the propagation of the first stimulation signal or the evoked electrical signal within the first tissue.

31. The method of claim 27, wherein the one or more stimulation parameters of the second stimulation signal comprises one or more of a signal width, a signal amplitude, or a signal frequency of the second stimulation signal.

32. The method of claim 27, further comprising:
determining, by the processing circuitry, a change in the sensed information representative of the evoked electrical signal; and
adjusting, by the processing circuitry, the one or more stimulation parameters of the second stimulation signal based on the determined change in the sensed information.

33. The method of claim 27, wherein the evoked electrical signal comprises an evoked compound action potential (ECAP) signal.

34. The method of claim 27, further comprising:
receiving, by the processing circuitry and from an external device in communication with the first IMD, an indication from the patient of the effect of the delivered second stimulation signal on a symptom;
adjusting, by the processing circuitry, the one or more stimulation parameters of the second stimulation signal based on the received indication; and
controlling, by the processing circuitry, the stimulation generation circuitry to deliver the second stimulation signal to the first tissue according to the adjusted one or more stimulation parameters.

35. A computer-readable medium comprising instructions that, when executed, cause processing circuitry of a first implantable medical device (IMD) to:
sense, via sensing circuitry of the first IMD implanted within a first tissue of a first region of a patient, information representative of an evoked electrical signal elicited by a first stimulation signal delivered from a second IMD different from the first IMD to a second tissue of a second region of the patient;
determine, based on the sensed information representative of the evoked electrical signal, one or more stimulation parameters at least partially defining a second stimulation signal; and
control stimulation generation circuitry of the first IMD to deliver the second stimulation signal to the first tissue according to the one or more stimulation parameters.
